(19) United States
(12) Patent Application Publication
(10) Pub. No.: US 2026/0083936 A1
(43) Pub. Date: Mar. 26, 2026

(54) LOCKING HUB AND RELATED SYSTEMS AND METHODS

(71) Applicant: Merit Medical Systems, Inc., South Jordan, UT (US)

(72) Inventor: Christopher Cindrich, Highland, UT (US)

(21) Appl. No.: 19/334,450

(22) Filed: Sep. 19, 2025

Related U.S. Application Data
(60) Provisional application No. 63/697,193, filed on Sep. 20, 2024.

(51) Int. Cl. A61M 25/00; A61M 25/09

(52) U.S. Cl. CPC A61M 25/0097; A61M 25/09; A61M 2025/09075; A61M 2025/09125

(57) ABSTRACT

A locking mechanism is provided including a proximal component including a distal end including a first spanning distance and a gap disposed between a material of the distal end defining the first spanning distance. A distal component is rotationally coupled with the proximal portion. The distal component includes a cavity including a distal end, the cavity rotationally coupled with the distal end of the proximal component. The cavity includes a second spanning distance of the distal end of the cavity. A rotation of the proximal component with respect to the distal component, or the distal component with respect to the proximal component, is configured to align a direction of the first spanning distance with a direction of the second spanning distance and diminish the gap of the proximal component.

### RELATED CASES

This application claims priority to U.S. Provisional Application No. 63/697,193, filed on Sep. 20, 2024, and titled “LOCKING HUB AND RELATED SYSTEMS AND METHODS,” which is hereby incorporated by reference in its entirety.

### TECHNICAL FIELD

The present application relates to stent prostheses, guidewires, catheters, and methods of using the same. More particularly, some embodiments relate to a locking hub.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 illustrates a perspective view of a catheter system, according to some embodiments described herein.

FIG. 2 illustrates a perspective view of a hub assembly of the catheter system of FIG. 1, according to some embodiments described herein.

FIG. 3 illustrates a perspective view of the hub assembly of the catheter system of FIG. 1, according to some embodiments described herein.

FIG. 4 illustrates a perspective view of the locking hub of the catheter system of FIG. 1, according to some embodiments described herein.

FIG. 5 illustrates an exploded view of the locking hub of the catheter system of FIG. 1, according to some embodiments described herein.

FIG. 6 illustrates a top view of a proximal component of the locking hub of the catheter system of FIG. 1, according to some embodiments described herein.

FIG. 7A illustrates an anterior view of a first embodiment of the proximal component of the locking hub of the catheter system of FIG. 1, according to some embodiments described herein.

FIG. 7B illustrates an anterior view of the proximal component of FIG. 7A in a closed configuration, according to some embodiments described herein.

FIG. 7C illustrates a perspective, cutaway view of a third embodiment of the proximal component of FIG. 7A, according to some embodiments described herein.

FIG. 7D illustrates a perspective, cutaway view of a fourth embodiment of the proximal component of FIG. 7A, according to some embodiments described herein.

FIG. 8 is a top view of a distal component of the locking hub of the catheter system of FIG. 1, according to embodiments described herein.

FIG. 9A illustrates a posterior view of a distal component of the locking hub of the catheter system of FIG. 1, according to some embodiments described herein.

FIG. 9B illustrates a perspective view of the distal component of FIG. 9A, according to some embodiments described herein.

FIG. 10A illustrates a lateral, cutaway view of an open configuration of the locking hub of the catheter system of FIG. 1, according to some embodiments described herein.

FIG. 10B illustrates a lateral, cutaway view of a closed configuration of the locking hub of the catheter system of FIG. 1, according to some embodiments described herein.

FIG. 11A illustrates a lateral, cutaway view of a second embodiment of the cantilevers of the proximal component of FIG. 6, according to some embodiments described herein.

FIG. 11B illustrates a lateral, cutaway view of the second embodiment of the cantilevers of FIG. 11A in a closed configuration, according to some embodiments described herein.

FIG. 12A illustrates a lateral, cutaway view of a third embodiment of the cantilevers of the proximal component of FIG. 6., according to some embodiments described herein.

FIG. 12B illustrates a lateral, cutaway view of the third embodiment of the cantilevers of FIG. 12A in a closed configuration, according to some embodiments described herein.

FIG. 13A illustrates a lateral, cutaway view of a guidewire and the open configuration of the locking hub of the catheter system of FIG. 1, according to some embodiments described herein.

FIG. 13B illustrates a lateral, cutaway view of a guidewire and the closed configuration of the locking hub of the catheter system of FIG. 1.

FIG. 14A illustrates a perspective view of a proximal component and a clip of the locking hub of the catheter system of FIG. 1, according to some embodiments described herein.

FIG. 14B illustrates a top view of the proximal component and the clip of FIG. 14A, according to some embodiments described herein.

FIG. 14C illustrates a perspective view of the clip of FIG. 14A, according to some embodiments described herein.

FIG. 14D illustrates a side view of the clip of FIG. 14C, according to some embodiments described herein.

FIG. 14E illustrates a top view of the proximal component and the clip of FIG. 14A engaging with a guidewire, according to some embodiments described herein.

FIG. 15 illustrates a lateral cutaway view of the proximal component of FIG. 15E with an elongated member (e.g., guidewire GW) including a core and a coating, secured by the proximal component, according to embodiments described herein.

FIG. 16A illustrates a detailed view of a hub assembly of a catheter system of with a locking hub in a first position, according to some embodiment described herein.

FIG. 16B illustrates a cross-sectional view of the locking hub in the first position of the hub assembly of FIG. 16A along line 16B-16B, according to some embodiments described herein.

FIG. 16C illustrates a cross-sectional view of the locking hub in the first position of the hub assembly of FIG. 16A along line 16C-16C, according to some embodiments described herein.

FIG. 16D illustrates a detailed view of the hub assembly of FIG. 16A with the locking hub in a second position, according to some embodiment described herein.

FIG. 16E illustrates a cross-sectional view of the locking hub in the second position of the hub assembly of FIG. 16D along line 16E-16E, according to some embodiments described herein.

FIG. 16F illustrates a cross-sectional view of the locking hub in the second position of the hub assembly of FIG. 16D along line 16F-16F, according to some embodiments described herein.

FIG. 17 illustrates a perspective view of a locking hub system according to some embodiments described herein.

FIG. 18 illustrates an exploded view of the locking hub system of FIG. 17, according to some embodiments described herein.

### DETAILED DESCRIPTION

Dilator systems and/or dual-wire lumen systems may be configured to deliver a guidewire from one branch of a branching stent prosthesis that is deployed within a patient's body to another branch. Such systems may be configured to be advanced from an insertion site external to an anatomical system to stent prosthesis internal to the anatomical system. A dual-wire lumen system may be deployed from an insertion site into a branching stent prosthesis. Such systems may be configured to deliver one guidewire with the assistance of another guidewire.

Stent prostheses may be deployed in various body lumens for a variety of purposes. Stent prostheses may be deployed, for example, in the arterial system for a variety of therapeutic purposes including the treatment of occlusions within the lumens of that anatomical system. For instance, stent prostheses may be designed for, and deployed within, the central venous system, peripheral vascular system, abdominal aortic aneurism system, bronchial system, esophageal system, biliary system, or any other system of the human body. Furthermore, additional prosthesis (e.g., such as grafts) may be similarly placed.

While navigating a guidewire, it may be beneficial to temporarily fix, or secure, a guidewire in place with respect to rotation and/or translation. This can ensure the stability and accuracy of a guidewire's position, preventing unintended movement (e.g., that could compromise the efficacy or success of a procedure). Reliably securing a guidewire is a tool healthcare providers (HCPs) can use to maintain control while navigating through anatomical pathways and delivering treatment.

A number of conventional methods and mechanisms exist to hold a guidewire in place during medical procedures. One common approach involves use of a clamping mechanisms, which exerts force on the guidewire to secure it in position. Clamps are often manually operated or spring-loaded. Another method can involve threaded locking mechanisms, where a screw or nut is tightened to apply force and fix a guidewire in place. Additionally, spring-loaded pins can be employed to hold the guidewire in place by engaging with small indentations or grooves along its surface.

Several challenges and limitations persist in securing a guidewire via conventional methods. One such challenge is the need for continuous adjustment and monitoring to maintain the desired level of stability throughout the procedure. Often, conventional mechanisms require readjustment to accommodate changes in the guidewire's position or patient movement, consuming valuable time and effort. Similarly, some mechanisms may be prone to over-or under-tightening, potentially causing damage to the guidewire or surrounding tissues. For instance, limitations in grip strength with spring-loaded pins may result in inadequate fixation, especially in challenging anatomical locations or with certain types of guidewires.

Additional challenges include the difficulty and rapidity of engaging or disengaging a device's locking or fixing mechanism. This challenge is particularly inherent with threaded locking mechanisms, which may require significant time and effort to adjust appropriately. Moreover, a lack of feedback mechanism indicating when a locking mechanism is sufficiently tightened adds to the potential risk of over-tightening or under-tightening. Users may struggle to determine the optimal level of force required to secure the guidewire effectively, leading to uncertainty and potential errors, and compromising the stability of the guidewire. Furthermore, the need for excessive user engagement, such as the time, manual strength, and effort needed to tighten threaded locking mechanisms, can impede procedural efficiency and increase the risk of complications.

Aspects and implementations of the present disclosure address these and additional challenges by providing a locking mechanism for rotationally and translationally fixing a guidewire with enhanced rapidity, stability, accuracy, and reduced activation force.

In some embodiments, a locking hub is provided that is capable of rotationally and translationally securing a guide wire to the hub via rotation of an actuating component. In some embodiments, the locking hub can include a fixing portion including dual, opposing cantilevers that press, or clamp, together upon rotation of the actuating component. Once pressed, the dual cantilevers can fix a guide wire in place with respect to the hub. In some cases, the rotation required to activate the fixing portion, or clamp the dual cantilevers, can be a quarter turn.

In some cases, the dual cantilevers can include a distal end that includes a major diameter and a minor diameter (e.g., an end that is ovular in shape). In some embodiments, the distal end can be rotated (e.g., within a cavity or receptacle) such that the major diameter is compressed. Through such compression, the dual cantilevers of the fixing portion can deflect to press, or clamp, onto an associated guide wire.

In some cases, the cross-sectional shape of the distal end of the fixing portion can include a major diameter and a minor diameter, and the receiving cavity can similarly include a distal end that includes a major diameter and a minor diameter. During operation, when the hub is in an uncompressed, or open, configuration, the major and minor diameters of the two distal ends may align. To activate the compressed, or closed, configuration, a user may rotate the fixing portion within the cavity such that the major diameter of the distal end of the fixing portion aligns with the minor diameter of the cavity. This can constrict the major diameter, and the fixing portion may clamp, or close, onto an associated guidewire.

In some embodiments, the hub may include a holding, or snapping, mechanism, to hold or snap the hub between open and closed configurations. In some embodiments, the holding mechanism can also provide tactile and/or audible feedback to a user of the device. A holding mechanism can be or include protrusions within a rotating portion of the hub which snap into corresponding detents within an opposing portion of the hub.

Aspect and implementations of the present disclosure provide advantages over conventional methods by providing a locking hub that securely and accurately fixes the translation and rotation of a guide wire. Additionally, the locking hub may be engaged or disengaged from an open/closed configuration via a quarter turn, enabling efficiency and enhancing convenience of use. Furthermore, visual, tactile, and/or auditory feedback can be provided to the user, enhancing security and confirmation of deployment of the fixing mechanism, while preventing over-clamping and limiting damage to guidewires, associated patient tissue, and further involved mechanisms and machinery.

The components of the embodiments as generally described and illustrated in the figures herein can be arranged and designed in a wide variety of different configurations. Thus, the following more detailed description of various embodiments, as represented in the figures, is not intended to limit the scope of the present disclosure but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical, fluidic and/or thermal interaction. Thus, two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., mounting hardware or an adhesive). The phrase “fluid communication” is used in its ordinary sense and is broad enough to refer to arrangements in which a fluid (e.g., a gas or a liquid) can flow from one element to another element when the elements are in fluid communication with each other.

The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

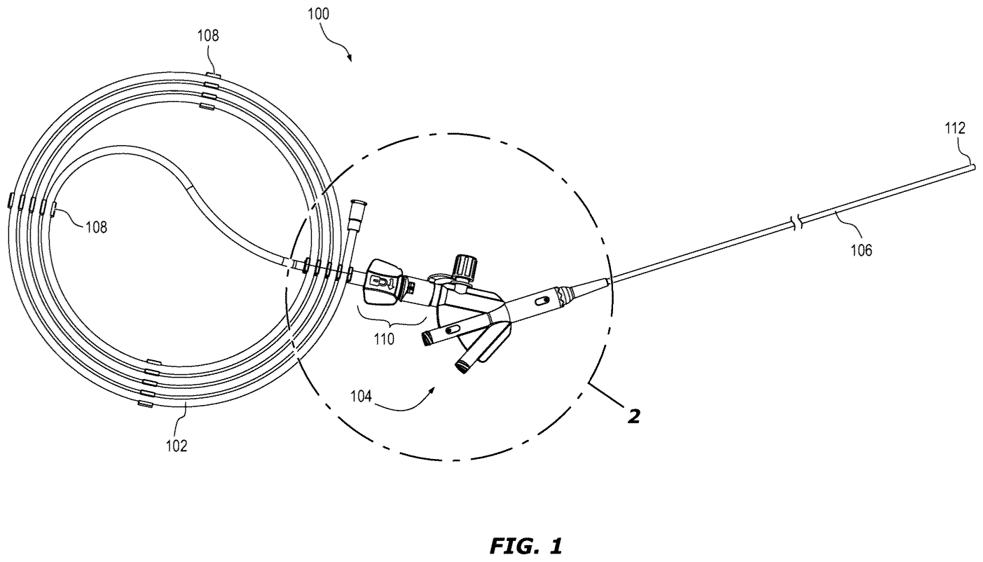

FIG. 1 illustrates a perspective view of a catheter system 100, according to some embodiments described herein. In the illustrated embodiment, the catheter system 100 includes a sheath 106 in fluid communication with a hub assembly 104. In certain embodiments, a coiled member 102 may be fluidly connected to the sheath 106 via the hub assembly 104. In some embodiments, coiled member 102 can be a hollow, or lumen-containing, shaft that is flexible and may be configured to retain a guidewire or other member with the hollow shaft. Coiled member 102 can be coiled and held together via clips 108. Coiled member 102 can be fluidly connected to a proximal side of hub assembly 104. Sheath 106 can be fluidly connected to a distal side of the hub assembly 104. In some embodiments, sheath 106 can extend distally from a distal end of the hub assembly 104, to a distal end 112, or delivery end, of catheter system 100. Hub assembly 104 can provide a proximal user input, with one or more components configured to allow a practitioner to deploy or otherwise manipulate further devices (e.g., a prosthesis, guidewire, etc.) disposed within catheter system 100.

During use, hub assembly 104 can be disposed outside of a patient's body, while sheath 106 can be advanced to a treatment location within the patient's body. For example, sheath 106 may be advanced from an insertion site (such as, for example, a femoral or jugular insertion site) to a treatment location within the vasculature. Catheter system 100 and/or sheath 106 can be configured to be advanced through bends, turns, or other structures within the anatomy of the vasculature.

In some embodiments, sheath 106 can include a braided wire frame and an impermeable material. In some cases, the braided wire frame can be of varying levels of rigidity. For example, a distal portion of the wire frame can have a greater pics/inch braid and/or rigidity than a proximal portion of the wire frame.

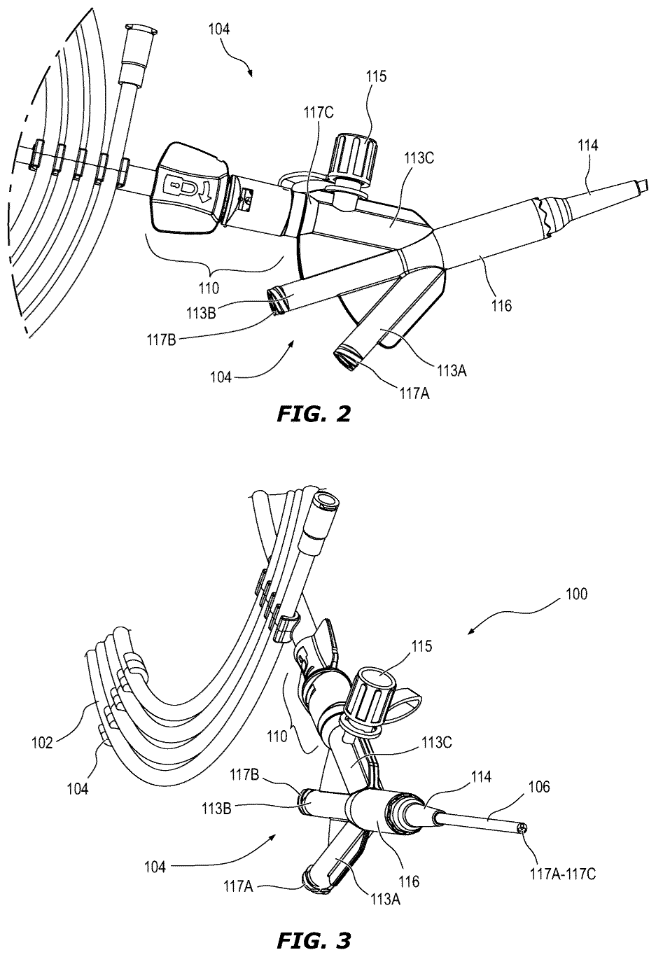

FIG. 2 to FIG. 3 illustrate perspective views of the catheter system of FIG. 1 and will be described jointly. Accordingly, FIG. 2 illustrates a detailed perspective view of hub assembly 104 of the catheter system of FIG. 1, according to some embodiments described herein. FIG. 3 illustrates a perspective view of hub assembly 104 of the catheter system of FIG. 1, according to some embodiments described herein.

In some embodiments, hub assembly 104 can include an internal cavity defined by one or more fluid pathways (e.g., fluid pathways 113A-113C) of hub assembly 104. Fluid pathways 113A-113C can include an internal lumen and extend from a distal end 114 of hub assembly 104 to a proximal end of hub assembly 104. In some embodiments, fluid pathways 113A-113C can fluidly connect, e.g., transfer fluid, from a proximal, connection end (e.g., connection portions 117A-117C) of a fluid pathway to distal end 114.

In some embodiments, fluid pathways 113A-113C can fluidly connect, or pass through, a main body 116. For example, each fluid pathways 113A-113C can include one or more internal lumens, fluidly connected to the fluid pathway of main body 116 of hub assembly 104 (and/or fluidly connect to corresponding, distinct, lumens 117A-117C within sheath 106). Sheath 106 may include one, two, or any number of internal lumens (e.g., lumens 117A-117C) fluidly connect to, or through, fluid pathways 113A-113C. Thus, fluid pathways 113A-113C, corresponding connection portions, and lumens 117A-117C within sheath 106 can be fluidly connected and be used to transfer fluids from a proximal end to a distal end of catheter system 100 (or vice-versa). Similarly, in some embodiments, one or more elongate members (e.g., guidewires, inflation lumens, etc.) can be passed through catheter system 100 from a proximal end to a distal end (or vice-versa). In some embodiments, any number of fluid pathways, corresponding connection portions, and lumens can be included in hub assembly 104.

In some embodiments, hub assembly 104 can further include a side port 115 fluidly connected to a fluid pathway of hub assembly 104. Side port 115 can include a cap for closing port and/or may include one or more valves. In some embodiments side port 115 may be used to flush one or more portions of hub assembly 104.

Hub assembly 104 can further include a locking hub 110 of hub assembly 104 can be used to lock, or fix, an elongate member (e.g., a guidewire) passing through an interior bore of hub assembly 104. For instance, in some cases, a guide wire can be passed through a proximal end of the hub assembly through fluid pathway 113C and out sheath 106 (e.g., through one of the lumens of lumens 117A-117C) into the vasculature of a patient. Locking hub 110 can be employed by a user of catheter system 100 to fix, or lock, such a guidewire in place. Locking hub 110 may rotationally and translationally fix a guidewire, with respect to hub assembly 104.

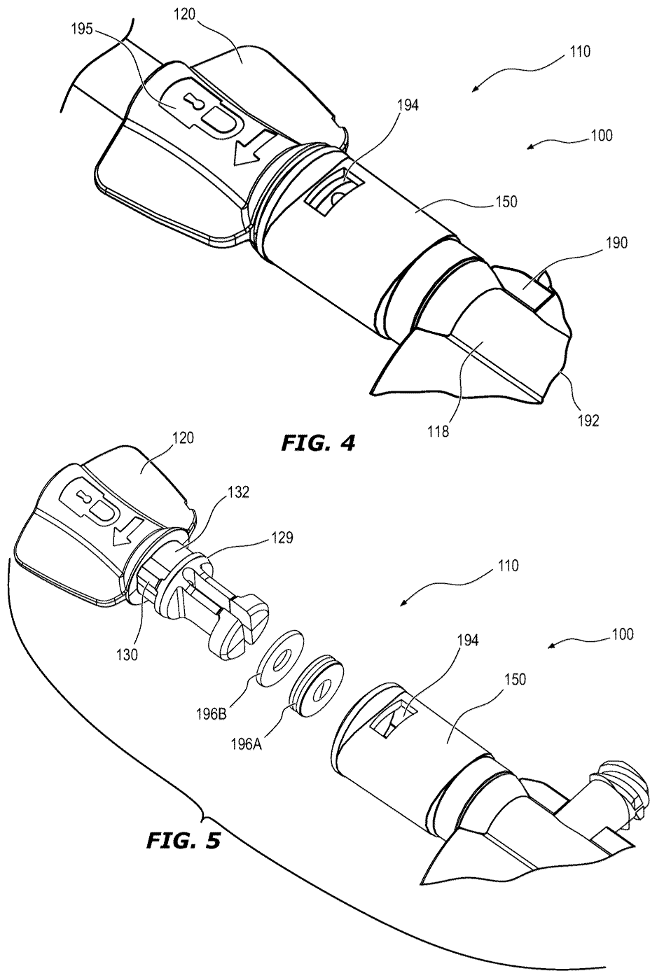

FIG. 4 illustrates a perspective view of locking hub 110 of catheter system 100 of FIG. 1, according to some embodiments described herein. Locking hub 110 can include a proximal component 120 and a distal component 150

Distal component 150 can include one or more windows 194 which create aspects of a detent means discussed in more detail below. Proximal component 120 can include visible indicia 195 to signal to a user of the device a direction for rotation used to lock locking hub 110.

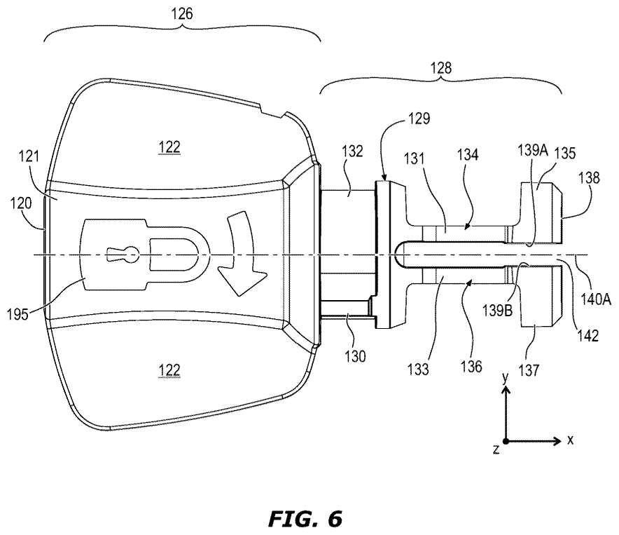

FIG. 5 illustrates an exploded view of locking hub 110, according to some embodiments described herein. Proximal component 120 and distal component 150 are visible and will be described in further detail with respect to FIG. 6 to 9. In some embodiments, a valve member 196A and a washer 196B can be used to reduce fluid leaking between proximal component 120 and distal component 150.

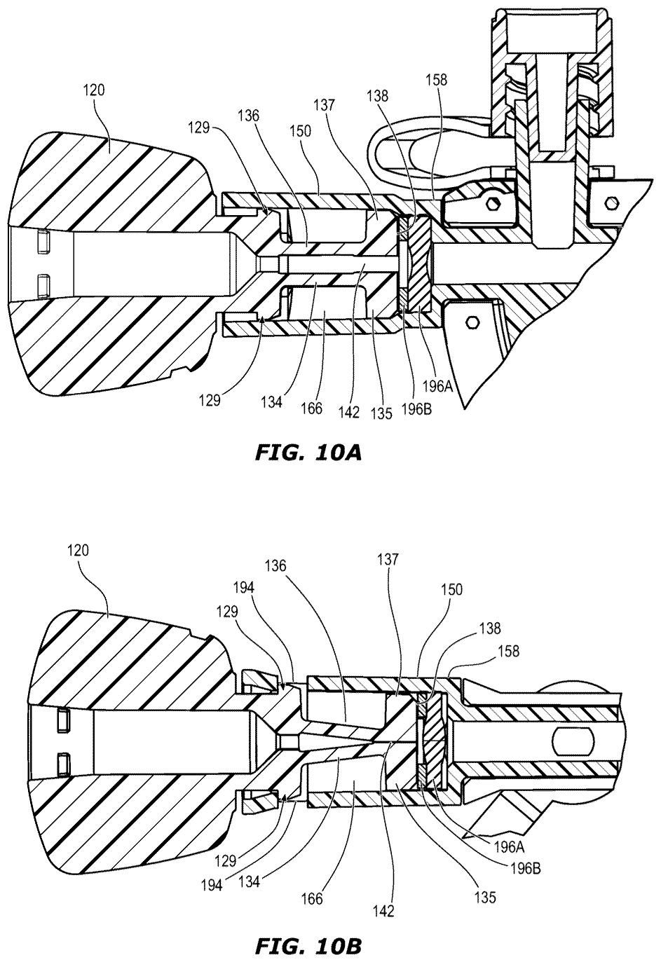

Proximal component 120 of locking hub 110 may include a plurality of first detents 130. First detents 130 extend radially outward from cylindrical portion 132. For example, proximal component 120 may include cylindrical portion 132 with includes one or more first detents 130. In the illustrated embodiment of FIG. 5, a single first detent 130 is illustrated, but cylindrical portion 132 may include two or more first detents 130. Cylindrical portion 132 may further include a rim 129 that is configured to engage with windows 194. In other words, as proximal component 120 is inserted into distal component 150, rim 129 engages with windows 194 and secures proximal component 120 to distal component 150. This attachment may be irreversible. This engagement may be seen in FIG. 10B.

FIG. 6 illustrates a top view of proximal component 120 of locking hub 110 of catheter system 100 of FIG. 1, according to some embodiments described herein. Proximal component 120 can include an actuating portion 126 and a fixing portion 128.

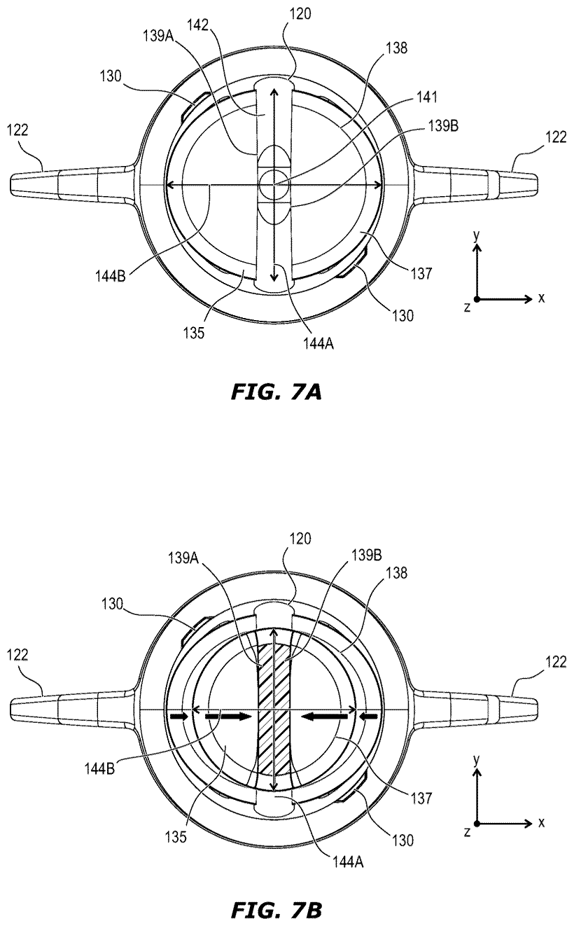

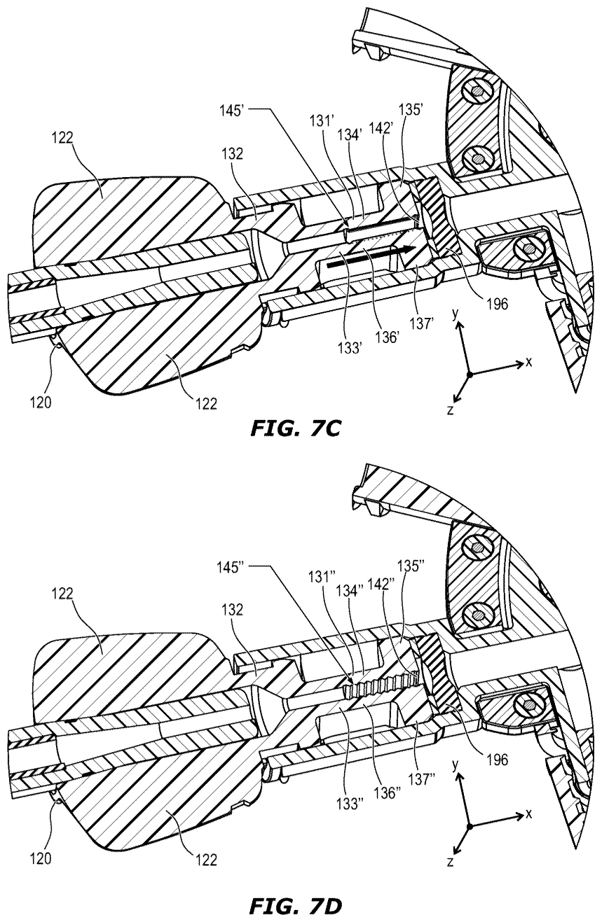

Actuating portion 126 can include a main body 121 and flanges 122. Main body 121 can be substantially cylindrical and include a bore (e.g., bore 141 seen in FIG. 7A) or hollow portion that extends along a longitudinal axis 140A of main body 121. Flanges 122 can extend radially from proximal component 120. Actuating portion 126 can be used by a user of locking hub 110 to rotate, or torque, the actuating portion 126 (e.g., with respect to locking hub 110 or other components of locking hub 110).

Fixing portion 128 can include cylindrical portion 132, a first cantilever portion 134 and a second cantilever portion 136. In some embodiments, cylindrical portion 132 can be placed between the proximal actuating portion 126 and the distal fixing portion 128. Cylindrical portion 132 can be substantially cylindrical and include one or more first detents 130. First detents 130 can be used to detain, or secure, a rotational orientation and limit travel of proximal component 120 with respect to locking hub 110 or other components of locking hub 110.

First cantilever portion 134 can include a gripping portion 139A and second cantilever portion 136 can include gripping portion 139B. Gripping portions 139A-139B can extend an entire inner longitudinal distance of the cantilevers, although in some embodiments, gripping portions 139A-139B can only extend a partially longitudinal distance, such as the longitudinal distance corresponding to cantilever heads 135 and 137. In some embodiments, gripping portions 139A-139B only extend a small inner longitudinal distance of first cantilever portion 134 and second cantilever portion 136.

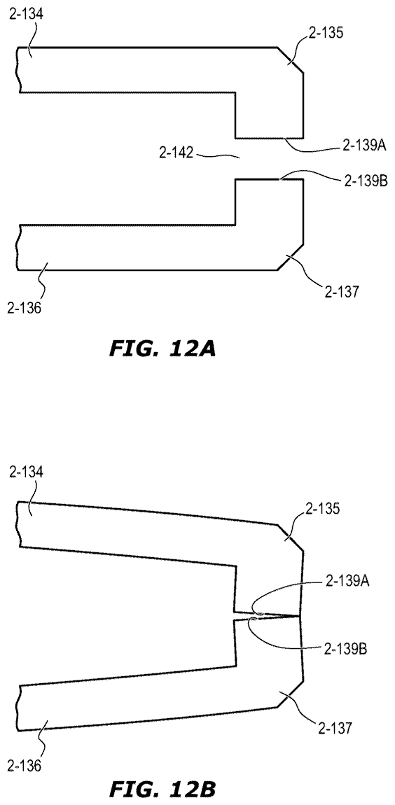

As will be further described with respect to FIG. 12A to FIG. 14B, cantilever portions 134 and 136 can bend when force is exerted onto them. For instance, first cantilever portion 134 and second cantilever portion 136 can be separated via gap 142. When forced towards longitudinal axis 140A, first cantilever portion 134 and second cantilever portion 136 can deflect such that cantilever heads 135 and 137 displace to axis 140A. Gripping portions 139A-139B can approach, and in some cases, contact each other. In such a way, gripping portions 139A-139B can be made to grip an elongate member (e.g., a guidewire) disposed within bore 141 of locking hub 110.

Cantilever heads 135 and 137 can together form a distal face 138 (as will be further described in FIG. 7A). Cantilever heads 135 and 137 can extend a depth, or a distance in the X axis. Distal face 138 defines a first spanning distance that includes gap 142.

Cantilever portions 134 and 136 can include cantilever beams 131 and 133 that are substantially thinner than cylindrical portion 132. Cantilever beams 131 and 133 can be resilient and deflectable, facilitating deflection of cantilever heads 135 and 137.

FIG. 7A illustrates an anterior view of a first embodiment of proximal component 120 of locking hub 110 of the catheter system of FIG. 1, according to some embodiments described herein. In the illustrated embodiment, distal face 138 can be formed by cantilever heads 135 and 137. Cantilever heads 135 and 137 can be separated by gap 142. Bore 141 can be seen extending through proximal component 120. When cantilever heads 135 and 137 are made to deflect, gap 142 can be diminished.

Gripping portions 139A-139B can be an interior portion of cantilever heads 135 and 137, respectively. Gripping portions 139A-139B can be configured to mate together when cantilever heads 135 and 137 are brought together. For instance, gripping portions 139A-139B can approach each other, closing gap 142, when pushed closer together (e.g., when locking hub 110 is transitioned into a closed configuration). Otherwise stated, gripping portions 139A-139B can come closer together in the X direction, and gap 142 can be diminished or removed.

In some embodiments, gripping portions 139A-139B can be parallel, straight edges, or surfaces configured to mate together. For instance, gripping portions 139A-139B can extend through a depth of cantilever heads 135 and 137. E.g., in the X direction as seen in FIG. 6; e.g., in the negative Z direction of FIG. 7A (or into the page). In some embodiments, gripping portions 139A-139B can form a groove, as will be further described with respect to FIG. 7C, when brought together. Such a groove can be aligned with bore 141 of locking hub 110 and can be configured to better grip an elongate member (e.g., a guidewire) passing through locking hub 110. In some embodiments, gripping portions 139A-139B may be a smooth surface. In some embodiments, gripping portions 139A-139B can include a surface or material coating (e.g., a polymer coating, an elastomeric coating, a tacky or gritty coating, a serrated surface, a beveled surface, etc.) to better secure, or grip, an elongate member (e.g., a guidewire) passing through locking hub 110.

In some embodiments, distal face 138 (which includes distal faces of cantilever heads 135 and 137 and gap 142 therebetween) in an unconstrained position can include a major diameter 144B and/or a minor diameter 144A. In some embodiments, distal face 138 may be of an ovular shape, or include a cross section that is of an ovular shape. Major diameter 144B and minor diameter 144A (e.g., an ovular or elliptical shape) can extend throughout cantilever heads 135 and 137.

FIG. 7B illustrates an anterior view of the proximal component 120 of FIG. 7A in a closed configuration, according to embodiments described herein. In the illustrated embodiment, the closed configuration can be seen and cantilever heads 135 and 137 can be pushed together to close, as indicated by the arrows of FIG. 7B. In some cases, a gap (e.g., gap 142 as described with respect to FIG. 7A) between the cantilever heads 135 and 137 can be closed. In alternate embodiments, gap 142 can be diminished, as opposed to closed. As illustrated in FIG. 7B, major diameter 144B decrease due to the closing of gap 142 while minor diameter 144A maintains its length.

FIG. 7C to FIG. 7D illustrate anterior and cutaway views of additional embodiments, respectively, of the proximal component of the locking hub of catheter system 100 of FIG. 1. FIG. 7C to FIG. 7D depict embodiments of a proximal component 120′ and 120″ that resembles proximal component 120 described with respect to FIG. 7A in certain respects. Accordingly, like features are designated with like reference numerals. Otherwise stated, elements from FIG. 7C to FIG. 7D can be the same, or similar, as similarly labeled elements of FIG. 7A. Elements that are different can be identified via a “prime” designation. For example, the embodiments depicted in FIG. 7C to FIG. 7D include a gap 142′ and 142″, that may, in some respects, resemble gap 142 of FIG. 7A, yet include varying features or embodiments thereof. Relevant disclosure set forth above regarding similarly identified features thus may not be repeated hereafter. Moreover, specific features of the proximal component of FIG. 7C to FIG. 7D, and related components shown in FIG. 7C to FIG. 7D, may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the embodiments of the proximal component and related components depicted in FIG. 7C to FIG. 7D. Any suitable combination of the features, and variations of the same, described with respect to the proximal component and related components illustrated in FIG. 7A can be employed with the embodiments of proximal components and related components of FIG. 7C to FIG. 7D, and vice versa. This pattern of disclosure applies equally to further embodiments depicted in subsequent figures and described hereafter, wherein the leading digits may be further incremented.

FIG. 7D illustrates a perspective, cutaway view of another embodiment of the proximal component 120′ of locking hub 110 of catheter system 100 of FIG. 1, according to some embodiments described herein. As seen in the illustrated embodiment, grooves 145′ include a cutout portion (e.g., forming a portion of, or being within, gap 142′). In the embodiment, grooves 145′ can extend only a minimal distance radially into cantilever portions 134′ and 136′. In some embodiments, grooves 145′ can extend only partially through cantilever portions 134′ and 136′ in the longitudinal direction. For instance, grooves 145′ may extend any distance though any portion of cantilever portions 134″ and 136″ and/or cantilever heads 135′ and 137′.

As discussed, grooves 145′ can serve to secure an elongate member (such as a guidewire) in an orientation or location with respect to locking hub 110.

FIG. 7E illustrates a cutaway perspective view of another embodiment of a proximal component 120″ of locking hub 110 of catheter system 100 of FIG. 1, according to some embodiments described herein. As seen in the illustrated embodiment, grooves 145″ include cutout portions and/or serrations distributed longitudinally along an inward face of cantilever portions 134″ and 136″ (e.g., forming a portion of, or being within, gap 142″). In some embodiments, grooves 145″ and/or serrations can extend only a minimal distance radially, or laterally, into cantilever portions 134″ and 136″. In some embodiments, grooves 145″ and/or serrations can extend only partially through cantilever portions 134″ and 136″ in the longitudinal direction. For instance, grooves 145″ can extend only throughout the material of the cantilever beams 131″ and 133″ of cantilever portions 134″ and 136″. In alternate embodiments, grooves 145″ may extend any distance though any portion of cantilever portions 134″ and 136″ and/or cantilever heads 135″ and 137″.

In some embodiments, grooves 145″ and/or serrations can serve to enhance the clamping or securement force provided by cantilever portions 134″ and 136″ onto an elongate member passing through locking hub 110. Otherwise stated, grooves 145″ and/or serrations as seen in FIG. 7E can enhance a frictional grip from cantilever portions 134″ and 136″ onto an elongate member passing through locking hub 110 against unwanted translation or rotation of the elongate member.

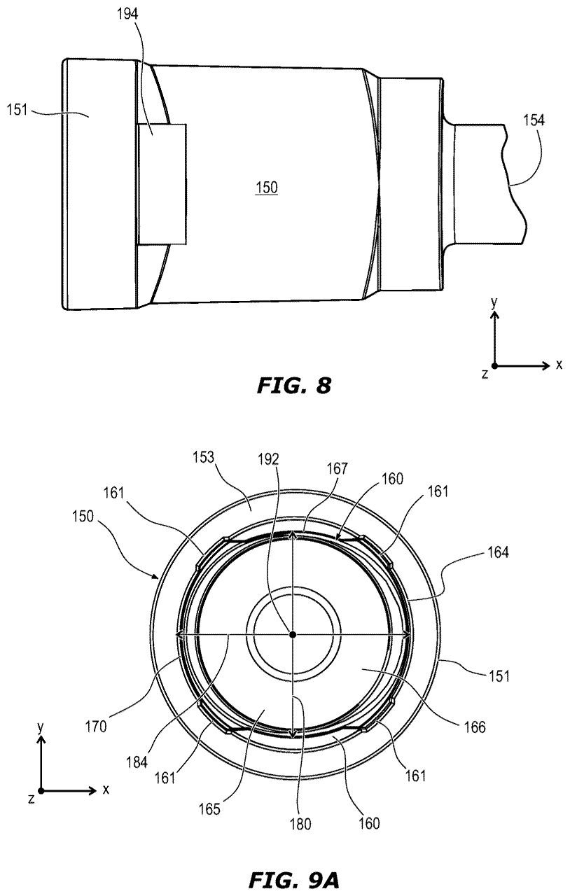

FIG. 8 is a top view of distal component 150 of locking hub 110 of the catheter system 100 of FIG. 1, according to embodiments described herein. Distal component 150 can include a main body 151.

In some embodiments, main body 151 and/or end portion 158 can be substantially cylindrical and include a void or cavity (not shown in FIG. 8) that extends along a longitudinal axis of distal component 150. An inside wall (not shown in FIG. 8) of the main body 151 and/or end portion 158 can define such a void or cavity. Main body 151 can receive cantilever heads 135 and 137 of proximal component 120 when locking hub 110 is assembled. In some cases, an interior void or cavity corresponding to end portion 158 can be of a different diameter or shape than an interior void or cavity corresponding to main body 151. In some cases, end portion 158 can receive valve member 196A and/or washer 196B of FIG. 5.

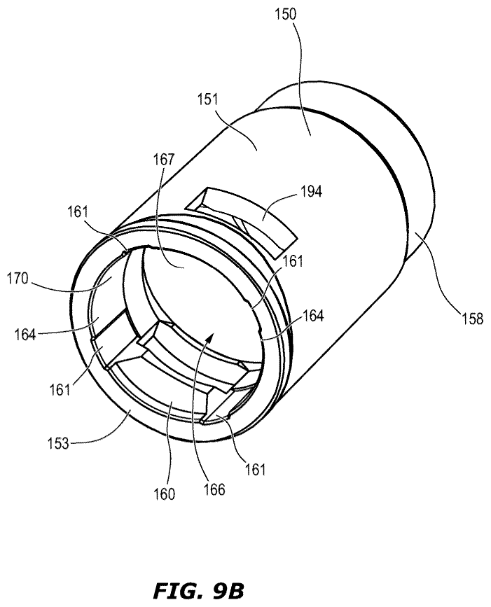

FIG. 9A illustrates a posterior view of distal component 150 of locking hub 110 of catheter system 100 of FIG. 1, according to some embodiments described herein. FIG. 9B illustrates a perspective view of distal component 150. In the illustrated embodiment, distal component 150 can include the cavity 166, bore 192, end face 165, and proximal face 153.

Cavity 166 can be substantially cylindrical and end at end face 165. In some cases, a cross section of cavity 166 can be substantially ovular. An inside wall (e.g., an inner wall 167) of main body 151 of distal component 150 (together with end face 165) can define cavity 166. Cavity 166 can be defined by inner wall 167 and be included within main body 151 of distal component 150. Inner wall 167 can extend along a longitudinal axis of distal component 150 e.g., along the Z axis of FIG. 9A (or into the page). Cavity 166 can be configured to receive fixing portion 128 of proximal component 120.

In some cases, end face 165 can be substantially circular, or substantially ovular or elliptical, in cross-sectional shape. In the illustrated embodiment, end face 165 is circular.

In some cases, cavity 166 can include a major diameter 184, a minor diameter 180, and/or an ovular cross-sectional shape (e.g., as seen in a cross-section of inner wall 167). In some cases, inner wall 167 and the defined cavity 166 can be substantially ovular, or elliptical, in shape. For instance, in some cases, major diameter 184, minor diameter 180, and/or an ovular shape can extend a longitudinal distance, e.g., along the Z axis of FIG. 9A (or into the page).

In some cases, an ovular, or elliptical shape, of end face 165 and/or inner wall 167 can extend for a portion, or all, of inner wall 167. For instance, in some embodiments, an ovular cross section of the cavity 166 can be disposed in a middle portion (with respect to the longitudinal distance) of cavity 166. In other embodiments, the ovular or elliptical section can be disposed at the start, or end, of cavity 166.

In other embodiments, the ovular or elliptical section of cavity 166 can be disposed at a portion of cavity 166 corresponding to a distal end of main body 151, and adjacent end portion 158 (of FIG. 8). The distal end of cavity 166 defines a second spanning distance. In some embodiments, the ovular or elliptical section of cavity 166 can be disposed along a portion of cavity 166 corresponding to main body 151 (as seen in FIG. 8). The portion of cavity 166 corresponding to end portion 158 (of FIG. 8) can be similarly ovular, or substantially circular.

In some embodiments, cavity 166 and end face 165 can be configured to mate with cantilever heads 135 and 137 of fixing portion 128 of proximal component 120. For instance, in some embodiments, major diameter 144B, minor diameter 144A, and/or ovular shape of distal face 138 (as seen in FIG. 7A) of cantilever heads 135 and 137 can match major diameter 184, minor diameter 180, and/or ovular shape of cavity 166.

As cantilever heads 135 and 137 and fixing portion 128 of proximal component 120 are rotated with respect to distal component 150, inner wall 167 of cavity 166 can constrict the major diameter 144B (seen in FIG. 7A) of distal face 138. This may gradually close gap 142 between cantilever heads 135 and 137, as the overall diameter of the space in which major diameter 144B of cantilever heads 135 and 137 must fit into is restricted. Once gap 142 has been diminished to a predetermined distance, or are contacting with a predetermined force, locking hub 110 can be determined to be in a closed configuration.

In some embodiments, in the closed configuration, major diameter 144B of distal face 138 of cantilever heads 135 and 137 may align with minor diameter 180 of cavity 166. As mentioned, this misalignment can constrict, or close, major diameter 144B (and gap 142) of distal face 138 of cantilever heads 135 and 137. This can deflect cantilever portions 134 and 136 such that cantilever heads 135 and 137 are moved closer together and gap 142 between them is diminished or closed. In the closed configuration, cantilever heads 135 and 137 or fixing portion 128 may grip, or secure, an elongate member (e.g., a guidewire) passing through bore 141 of locking hub 110.

Additionally, in an open configuration of locking hub 110, major diameter 144B of distal face 138 of cantilever heads 135 and 137 may align with major diameter 144B of cavity 166 (e.g., major diameter 184 along a portion or all of the cavity 166, inner wall 167, and/or end face 165). Minor diameters 144A and 180 of each proximal component 120 and distal component 150 may align as well. In the open configuration cantilever heads 135 and 137 may be relieved of inward force such that a larger, or predetermined space of gap 142 between cantilever heads 135 and 137 is present (or restored, if transitioning from a closed configuration to an open configuration).

To detain, or retain, an open or closed configuration of locking hub 110, proximal face 153 can engage with first detents 130 of cylindrical portion 132 (of FIG. 6 to FIG. 7A). For example, proximal face 153 can be configured to mate with cylindrical portion 132 (of FIG. 6 to FIG. 7A) of proximal component 120. Returning to FIG. 9A, proximal face 153 can include an interior circumference 170 with second detents 161, radial protrusions 160, and transition portions 164. Radial protrusions 160 can extend radially inward. Second detents 161 can extend radially outward. Transition portions 164 can connect second detents 161 and/or radial protrusions 160 of interior circumference 170.

Multiple depths, or magnitude of protrusion, for multiple second detents 161, radial protrusions 160, and/or transition portions 164 can exist on interior circumference 170. For instance, multiple, additional detents 161 (not seen in FIG. 9A) of lesser radial depth, when compared to second detents 161 (of FIG. 9A), can be disposed around transition portions 164. Thus, in some cases, proximal component 120 can click, or snap, as it is rotated with respect to distal component 150.

Interior circumference 170 can include one, more, or any combination of detents, protrusions, or transition portions. Interior circumference 170, second detents 161, radial protrusions 160, and/or transition portions 164 can be configured to engage, or frictionally engage, with detents (e.g., first detents 130 of FIG. 6) of proximal component 120.

In some embodiments, when proximal component 120 and distal component 150 are mated (as seen in FIG. 10A to FIG. 10B), first detents 130 of proximal component 120 can frictionally engage with interior circumference 170 of distal component 150. When rotated, first detents 130 (seen in FIG. 6 to FIG. 7A) can provide a frictional resistance to a user rotating proximal component 120. For instance, first detents 130 of proximal component 120 can rest, or lock, into second detents 161. In some cases, this may hold, or retain, proximal component 120 in a specific rotational position with respect to distal component 150. A certain, or specific, force may be required to overcome the hold or retention. In some cases, a user may have to overcome an initial requirement for rotational force to displace first detents 130 from within second detents 161. In some embodiments, an audible click may be heard when first detents 130 from within second detents 161.

In some cases, radial protrusions 160 can arrest, or stop, rotation of proximal component 120 with respect to distal component 150 by blocking passage of first detents 130 of the proximal component (e.g., radial protrusions 160 can constrict the diameter such that first detents 130 of proximal component 120 cannot pass). Functionally speaking, proximal component 120 may not be rotated past a certain point with respect to distal component 150.

In some cases, transition portions 164 of interior circumference 170 can increase, decrease, or “ramp” in diameter from a first end of transition portion 164 to a second end of the transition portion 164 (e.g., around the interior circumference 170).

Such a feature of transition portion 164 can provide a frictional resistance to a user of locking hub 110, resulting in tactile feedback indicating a rotational position of proximal component 120 and locking hub 110.

In some embodiments, any of the features (e.g., detents, protrusions, etc.) of interior circumference 170 can provide visual, tactile, and/or auditory feedback to a user of locking hub 110 (e.g., rotation of first detents 130 into a second detent 161 may provide an audible “click” to a user of locking hub 110 and/or removing first detents 130 from second detent 161 can provide tactile feedback that locking hub 110 is out of an detained position).

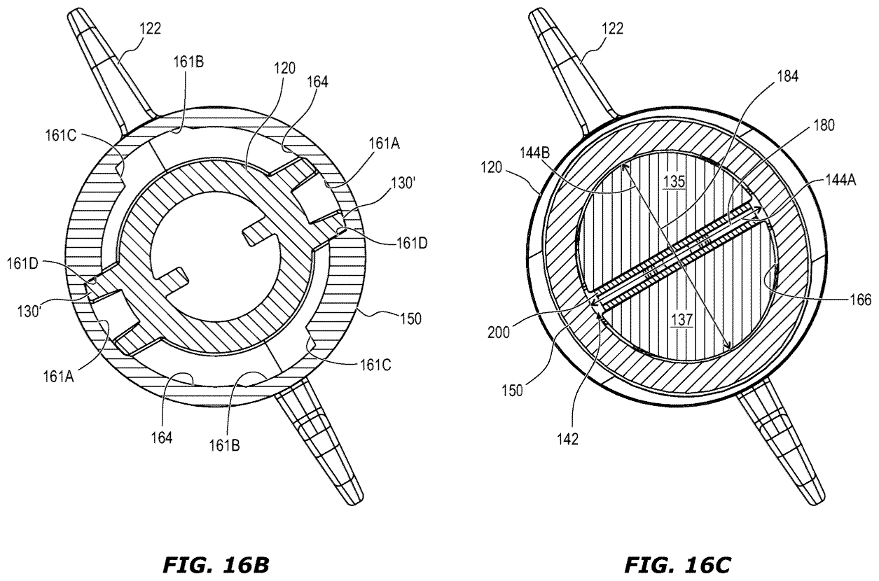

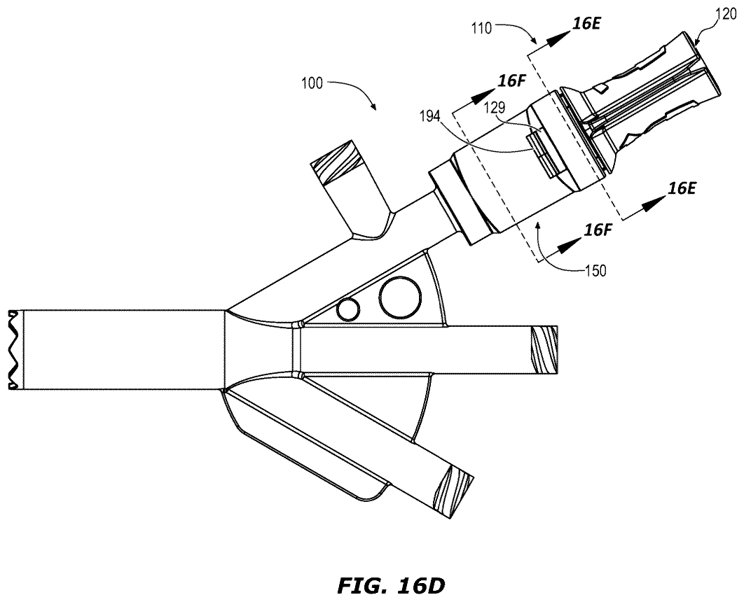

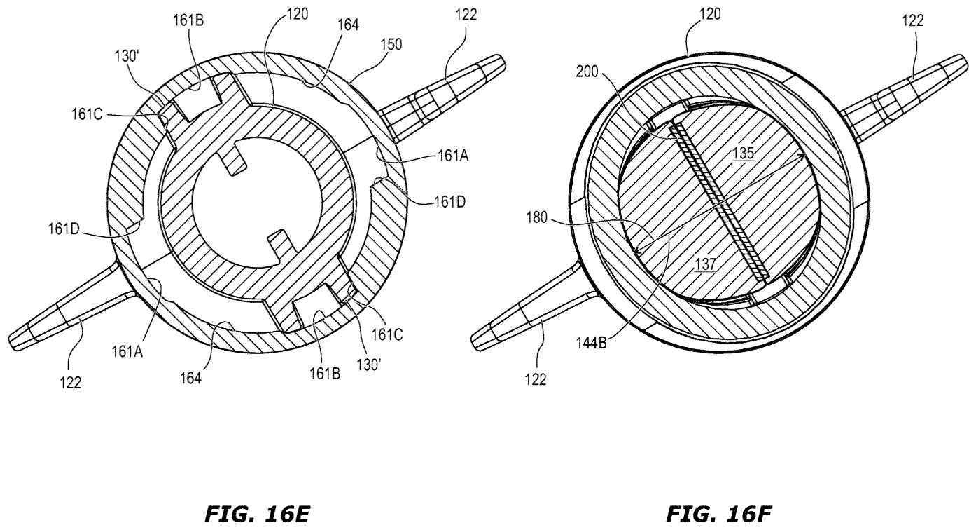

A description of twisting locking hub 110 from an open configuration or first position in which first detents 130 are engaged with second detents 161 to a closed configuration or second position in which first detents 130 are engaged with second detents 161 is detailed below in the description of FIG. 16A-16F.

FIG. 10A illustrates a cutaway, lateral view of an open configuration or first position of locking hub 110 of catheter system 100 of FIG. 1, according to some embodiments described herein. As seen in the illustrated embodiment, in the open configuration, cantilever heads 135 and 137 can be received within a portion of cavity 166. In the illustrated embodiment, distal component 150 and proximal component 120 and can be rotationally aligned such that the major and minor diameters 144B and 144A of cantilever heads 135 and 137 align with the major and minor diameters 184 and 180 of cavity 166. As shown, gap 142 can be present, and/or maximally open, in the open configuration.

As seen, valve member 196A and washer 196B are disposed within end portion 158 of distal component 150.

FIG. 10B illustrates a cutaway, lateral view of a closed configuration or second position of locking hub 110 of catheter system 100 of FIG. 1, according to some embodiments described herein. As seen in the illustrated embodiment, in the closed configuration, proximal component 120 and distal component 150 can be rotated such that major and minor diameters 144B and 144A of cantilever heads 135 and 137 do not align with major and minor diameters 184 and 180 of a portion of cavity 166. In the illustrated embodiment, gap 142 between two cantilever portions 134 and 136 and/or cantilever heads 135 and 137 can be diminished or constricted. As discussed, in some cases, gap 142 can be entirely closed within the closed configuration. This diminishment of the gap may fix, secure, or grip an elongate member (e.g., a guidewire) passing through a bore (e.g., bore 192) of locking hub 110. While only proximal component 120 is rotated relative to distal component 150, FIG. 10B illustrates proximal component 120 in the same position to illustrate the cantilever portions 134 and 136 in the constrained position.

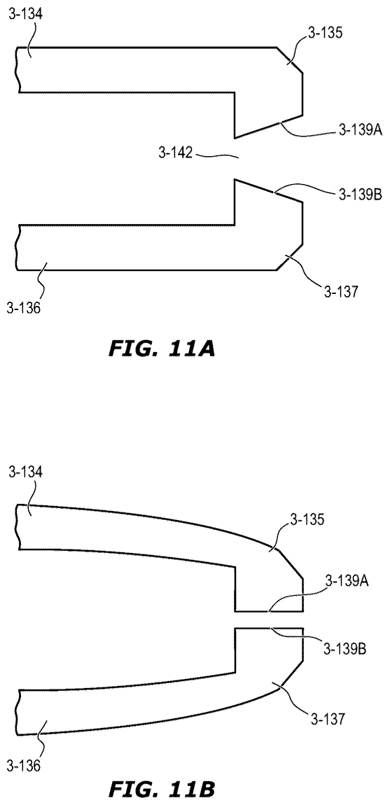

FIG. 11A to FIG. 12B illustrate lateral views of a second and third embodiment, respectively, of the cantilever portions 134 and 136 of proximal component 120 of FIG. 6. FIG. 11A to FIG. 12B depict embodiments of cantilevers that resemble the cantilevers described with respect to FIG. 6 in certain respects.

Accordingly, like features are designated with like reference numerals. Otherwise stated, elements from FIG. 11A to FIG. 12B can be the same, or similar, as similarly labeled elements of FIG. 6. Elements that are different can be identified via an increasing digit-prefix (e.g., “2-” or “3-”) designation. For example, the embodiments depicted in FIG. 11A to FIG. 12B include cantilever portion 2-134, cantilever portion 2-136, cantilever portion 3-134, and cantilever portion 3-136, that may, in some respects, resemble cantilever portions 134 and 136 of FIG. 6, yet include varying features or embodiments thereof. Relevant disclosure set forth above regarding similarly identified features thus may not be repeated hereafter. Moreover, specific features of the proximal component of FIG. 11A to FIG. 12B, and related components shown in FIG. 11A to FIG. 12B, may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the embodiments of the proximal component and related components depicted in FIG. 11A to FIG. 12B. Any suitable combination of the features, and variations of the same, described with respect to the proximal component and related components illustrated in FIG. 6 can be employed with the embodiments of proximal components and related components of FIG. 11A to FIG. 12B, and vice versa. This pattern of disclosure applies equally to further embodiments depicted in subsequent figures and described hereafter, wherein the leading digits may be further incremented.

FIG. 11A illustrates a cutaway lateral view of a second embodiment of cantilever portions 134 and 136 of proximal component 120 of FIG. 6, according to some embodiments described herein. In the illustrated embodiment, cantilever portion 2-134 and cantilever portion 2-136 are shown, with corresponding cantilever heads 2-135 and 2-137.

FIG. 11A shows cantilever portion 2-134 and cantilever portion 2-136 in an open configuration. Accordingly, gripping portion 2-139A and gripping portion 2-139B can be separated by a gap 2-142.

FIG. 11B illustrates a cutaway lateral view of the second embodiment of cantilever portion 2-134 and cantilever portion 2-136 of FIG. 11A in a closed configuration, according to some embodiments described herein. In the illustrated embodiment, cantilever portion 2-134 and cantilever portion 2-136 are shown, with corresponding cantilever heads 2-135 and 2-137.

When compared to the closed configuration of FIG. 10B, cantilever heads 2-135 and 2-137 can come together such that surfaces of gripping portion 2-139A and gripping portion 2-139B meet at an angle, instead of planarly. Otherwise stated, gripping portion 2-139A and gripping portion 2-139B can share a diminished contact surface, or point of contact, within the closed configuration (e.g., at a distal, terminal end seen in FIG. 11B). In some cases, this configuration can serve to enhancing the fixing, or gripping, ability of gripping portion 2-139A and gripping portion 2-139B onto an elongate member that is passed through locking hub 110.

FIG. 12A illustrates a cutaway lateral view of a third embodiment of cantilever portions 134 and 136 of proximal component 120 of FIG. 6., according to some embodiments described herein. In the illustrated embodiment, cantilever portion 3-134 and cantilever portion 3-136 are shown, with corresponding cantilever heads 3-135 and 3-137.

FIG. 12A shows cantilever portion 3-134 and cantilever portion 3-136 in an open configuration. Accordingly, gripping portion 3-139A and gripping portion 3-139B can be separated by a gap 3-142.

In some embodiments, gripping portion 3-139A and gripping portion 3-139B can include a planar surface disposed at an angle (e.g., not parallel to each other, as seen in FIG. 12A). In some cases, the opposing surfaces of gripping portion 3-139A and gripping portion 3-139B can serve to form a better grip, or fixing surface in the closed configuration, as will be further described with respect to FIG. 12B.

FIG. 12B illustrates a cutaway lateral view of the third embodiment of cantilever portion 3-134 and cantilever portion 3-136 of FIG. 12A in a closed configuration, according to some embodiments described herein. In the illustrated embodiment, cantilever portion 3-134 and cantilever 3-136 are shown, with corresponding cantilever heads 3-135 and 3-137.

When compared to the closed configuration of FIG. 10B and FIG. 11B, cantilever heads 3-135 and 3-137 can come together such that surfaces of gripping portion 3-139A and gripping portion 3-139B meet planarly, as opposed to at an angle. Otherwise stated, gripping portion 3-139A and gripping portion 3-139B can share an increased area of contact, within the closed configuration (e.g., as seen in FIG. 12B). In some cases, this configuration can serve to enhancing the fixing, or gripping, ability of gripping portion 3-139A and gripping portion 3-139B onto an elongate member that is passed through the device.

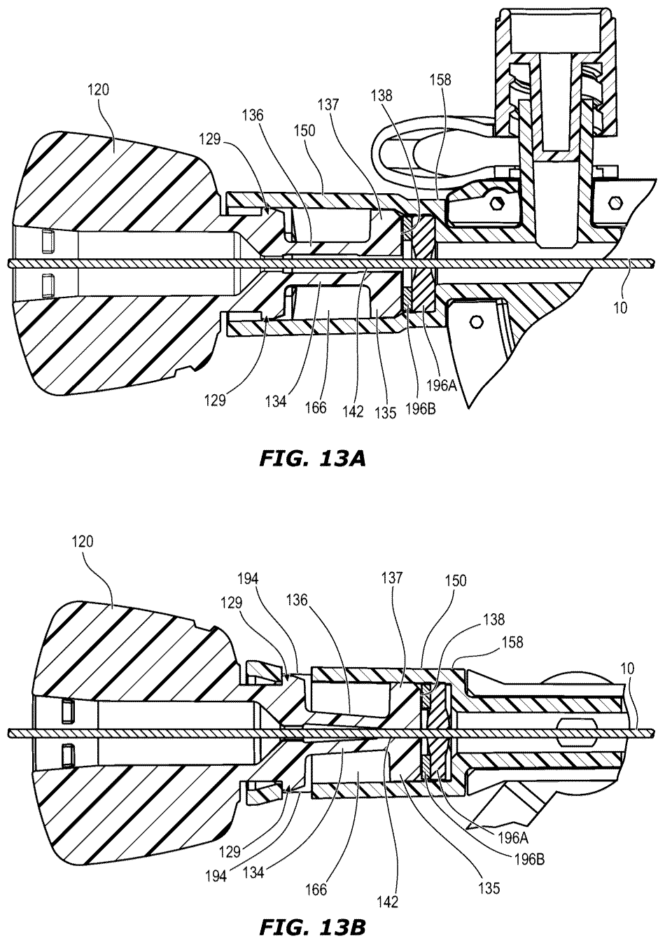

FIG. 13A illustrates a cutaway lateral view of a guidewire and the open configuration or first position of locking hub 110 of catheter system 100 of FIG. 1, according to some embodiments described herein. As seen in the illustrate embodiment, cantilever portions 134 and 136 and/or cantilever heads 135 and 137 can be separated, as a guidewire 10 passes through gap 142 between cantilever heads 135 and 137. Guidewire 10 can pass through bore 192 (and or the space where gap 142 would otherwise be).

FIG. 13B illustrates a cutaway lateral view of a guidewire and the closed configuration or second position of locking hub 110 of catheter system 100 of FIG. 1. As seen in the illustrate embodiment, the cantilevers portions 134 and 136 and/or cantilever heads 135 and 137 can be closed shut and secure, fix, and/or grip a guidewire 10 passing through locking hub 110. Guidewire 10 can pass through bore 192 (and or the space where gap 142 would otherwise be). While only proximal component 120 is rotated relative to distal component 150, FIG. 13B illustrates proximal component 120 in the same position to illustrate the cantilever portions 134 and 136 in the constrained position.

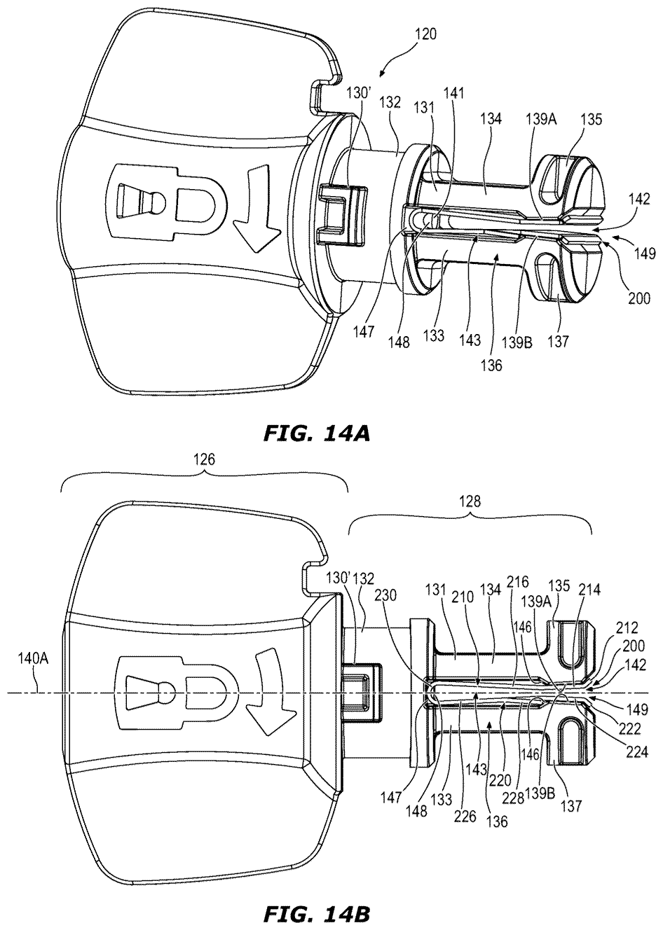

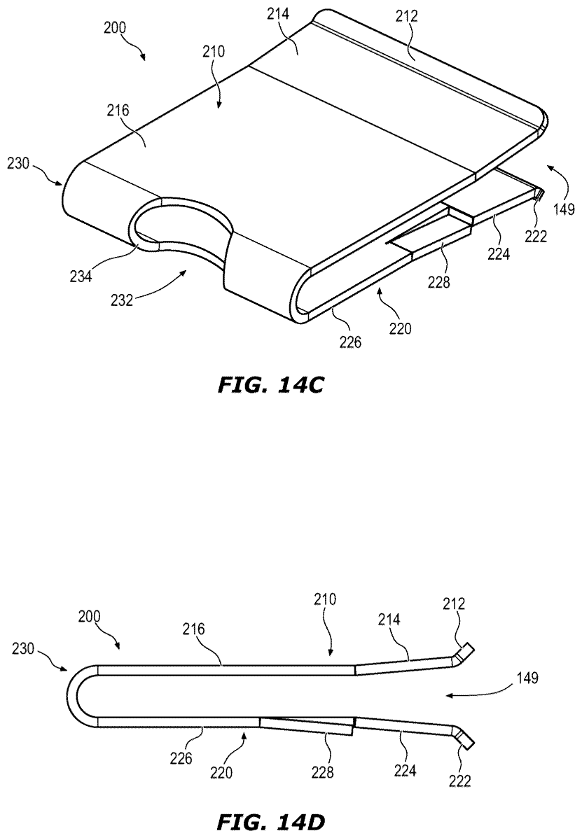

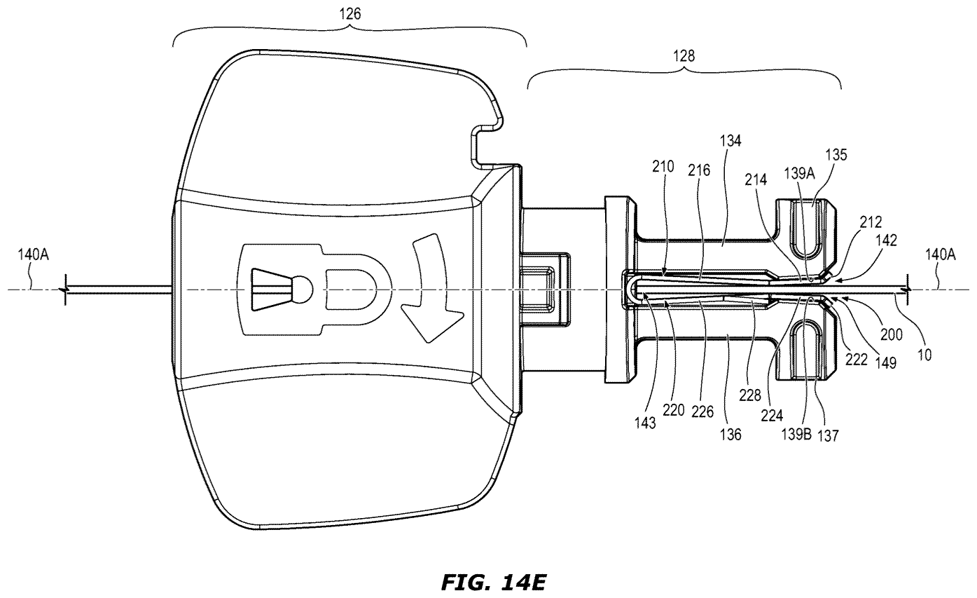

FIG. 14A-14B illustrate an embodiment of proximal component 120 further comprising a clip 200. Clip 200 is configured to provide additional securement to an elongate member (e.g., guidewire) disposed within bore 141 of locking hub 110. Clip 200 may be fabricated from a number of different materials, including metals. Exemplary metal materials include stainless steel, aluminum, galvanized steel, brass, copper, bronze, and the like.

FIG. 14A illustrates a perspective view of proximal component 120 of locking hub 110 of catheter system 100 of FIG. 1, according to some embodiments described herein. FIG. 14B illustrates a top view of proximal component 120. Proximal component 120 can include an actuating portion 126 and a fixing portion 128.

As discussed above, fixing portion 128 can include cylindrical portion 132, first cantilever portion 134 and second cantilever portion 136. Cylindrical portion 132 can include first detent 130′. First detent 130′ is shaped differently than first detent 130 of FIG. 5-7B. First detent 130′ is C-shape and is configured to engage with second detents 161 of distal component 150. First detent 130 and 130′ and second detents 161 may have a plurality of different shapes as long as first detents 130 and 130′ and second detent 161 are able to mate with each other.

First cantilever portion 134 can include gripping portion 139A and second cantilever portion 136 can include gripping portion 139B. Gripping portions 139A-139B can extend an entire inner longitudinal distance of cantilever portions 134 and 136, although in some embodiments, gripping portions 139A-139B only extend a partial longitudinal distance, such as the longitudinal distance corresponding to cantilever heads 135 and 137.

Cantilever portions 134 and 136 can bend when force is exerted onto them. For instance, first cantilever portion 134 and second cantilever portion 136 can be separated via gap 142. When forced towards longitudinal axis 140A, first cantilever portion 134 and second cantilever portion 136 can deflect such that cantilever heads 135 and 137 displace to axis 140A. Gripping portions 139A-139B can approach, and in some cases, contact each other. In such a way, gripping portions 139A-139B can be made to grip an elongate member (e.g., a guidewire) disposed within bore 141 of locking hub 110.

Cantilever portions 134 and 136 can include cantilever beams 131 and 133 that are substantially thinner than cylindrical portion 132. Cantilever beams 131 and 133 can be resilient and deflectable, facilitating deflection of cantilever heads 135 and 137.

As discussed above, fixing portion 128 may further include clip 200 disposed between cantilever portions 134 and 136. Clip 200 may be shaped so that clip 200 is disposed within a space 143 defined by the cantilever portions 134 and 136 and cantilever heads 135 and 137. Clip 200 floats within space 143 but is also constrained within space 143 by proximal component 120 such that clip 200 is secured within space 143. In other words, clip 200 is secured within space 143, but not by fixing clip 200 to first cantilever portion 134 and second cantilever portion 136 but not constraining clip 200 in space 143 by various components of proximal component 120. Clip 200 is slidable into space 143 and secured within space 143 after clip 200 is slid into space 143.

FIG. 14C-14D illustrates various view of clip 200. FIG. 14C illustrates a perspective view of clip 200 and FIG. 14D illustrates a side view of clip 200. Clip 200 comprises a first member 210, a second member 220, and a curved member 230 coupling first member 210 to second member 220. Clip 200 may comprise a spring that may return clip 200 to its original shape after being deformed by cantilever portions 134 and 136.

First member 210 comprises a first portion 212, a second portion 214, and a third portion 216. Second member 220 comprises corresponding features, a first portion 222, a second portion 224, and a third portion 226. First portion 212 of first member 210 is a free end that curves away from a longitudinal axis of clip 200 and first portion 222 of second member 220 is a free end that curves away from the longitudinal axis of clip 200 in an opposite direction of the free end of the first portion 212 of first member 210. Second portion 214 of first member 210 and second portion 224 of second member 220 are angled relative to each other. Second portion 214 of first member 210 may align with gripping portion 139A and second portion 224 of second member 220 may align with gripping portion 139B. Third portion 216 of first member 210 and third portion 226 of second member 220 may be parallel to each other. However, when clip 200 is inserted into space 143 as illustrated in FIG. 14B, third portion 216 of first member 210 and third portion 226 of second member 220 may be angled relative to each other due to the constraints of space 143.

Second portion 214 of first member 210 is disposed between first portion 212 and third portion 216. Second portion 224 of second member 220 is disposed between first portion 222 and third portion 226. Third portion 216 of first member 210 is coupled to curved member 230 and third portion 226 of second member 220 is coupled to curved member 230. In other words, third portion 216 of first member 210 is similar to first cantilever portion 134 and third portion 226 of second member 220 is similar to second cantilever portion 136.

Clip 200 may further comprise a second gap 149. Second gap 149 is disposed between gap 142 and is smaller than gap 142. As gap 142 is diminished by movement of cantilever head 135 and 137, second gap 149 is simultaneously diminished.

Second portion 224 of second member 220 comprises a pair of barbs 228 that are configured to engage with cantilever head 137. In other words, barbs 228 engage with an angled portion 146 of cantilever head 137. In the illustrated embodiment, each lateral side of second portion 224 of second member 220 have barbs. The curved member 230 is configured to engage with an inner wall 147 of space 143. Inner wall 147 may be curved similarly to curved member 230 of clip 200 as seen in FIG. 14B.

Accordingly, clip 200 is constrained within space 143 as barbs 228 engage with angled portion 146 and curved member 230 engages with inner wall 147, thereby prevent longitudinal movement of clip 200 as seen in FIG. 14B.

In some embodiments, second portion 214 of first member 210 may also comprise a pair of barbs 228 that are configured to engage with cantilever head 135. In other words, barbs 228 engage with an angled portion 146 of cantilever head 135.

The curved member 230 comprises an aperture 232. An elongate member (e.g., a guidewire) can pass longitudinally through the locking hub 110 through bore 192 and through aperture 232. Aperture 232 is large enough to fit around a ridge 148 of bore 141 (see FIGS. 14A and 14B for ridge 148). Ridge 148 extends from inner wall 147 into space 143. Accordingly, an edge 234 of aperture 232 does not engage with an elongate member (e.g., guidewire).

Aperture 232 may also engage with the perimeter of ridge 148. Accordingly, clip 200 is constrained within space 143 as aperture 232 engages with ridge 148 and thereby prevent lateral movement of clip 200 when disposed with space 143.

FIG. 14E illustrates a side view of proximal component 120 with an elongated member (e.g., guidewire 10) secured by proximal component 120. For example, when force is exerted onto cantilever portions 134 and 136, cantilever portions 134 and 136 can bend toward longitudinal axis 140A. In addition, clip 200 disposed within space 143 also deflects towards longitudinal axis 140A. First detent 130′ has a different shape from first detent 130 illustrated in FIG. 5.

For instance, first cantilever portion 134 and second cantilever portion 136 are separated via gap 142. When forced towards longitudinal axis 140A, first cantilever portion 134 and second cantilever portion 136 deflect such that cantilever heads 135 and 137 displace to axis 140A. This displacement also deflects second portion 214 of first member 210 and second portion 224 of second member 220 toward longitudinal axis 140A. Gripping portions 139A-139B can approach and the interior of second portion 214 of first member 210 and the interior of second portion 224 of second member 220, can contact each other. In such a way, gripping portions 139A-139B can be made to grip guidewire 10 disposed within bore 141 of locking hub 110 and extends through space 143 and gap 142.

In other words, clip 200 clamps down on guidewire 10 as cantilever portions 134 and 136 deflect toward longitudinal axis 140A. Clip 200 is clampable onto guidewire 10. The interior of second portion 214 of first member 210 and second portion 224 of second member 220 clamp down on guidewire 10 to secure it in place. In some embodiments, a portion of second portion 214 and third portion 216 of first member 210 and second portion 224 and third portion 226 of second member 220 engage with guidewire 10. In some embodiments, a length of second portion 214 and second portion 224 engage with guidewire 10.

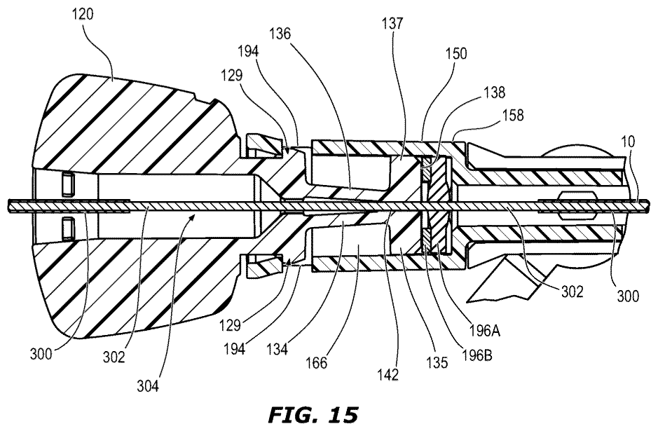

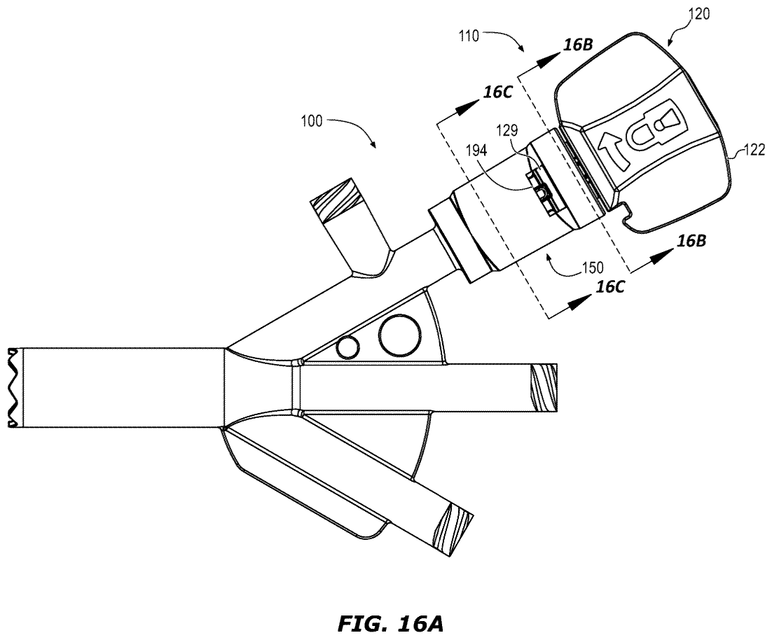

FIG. 15 illustrates a lateral cutaway view of proximal component 120 of FIG. 14E with an elongated member (e.g., guidewire 10) including a core 302 and coating 300, secured by proximal component 120, according to embodiments described herein. As previously described with respect to FIG. 14E, when force is exerted onto cantilever portions 134 and 136, cantilever portions 134 and 136 can bend toward longitudinal axis 140A. In some cases and/or alternatively, clip 200 disposed within space 143 also deflects towards longitudinal axis 140A.

In some embodiments, an elongate body of elongated member (e.g., guidewire 10) can be formed from a metallic core (e.g., core 302), which may be formed from elements such as stainless steel, nitinol, and so on. In some cases, the elongate body can be of a uniform diameter along its longitudinal length or axis. However, in some embodiments, elongated member (e.g., guidewire 10) can be of a non-uniform diameter. For instance, elongated member (e.g., guidewire 10) may be tapered or vary in diameter along its longitudinal axis to enhance maneuverability or be atraumatic when in use. For instance, in some cases, a distal end of elongated member (e.g., guidewire 10) can be tapered or of a cone-like, or similar, narrowing profile.

In some cases, an exterior surface of elongated member (e.g., guidewire 10) can include coating 300 (e.g., a PTFE or a hydrophilic polymer coating) disposed over metallic core 302 of the guidewire. For instance, in some cases, elongated member (e.g., guidewire 10) can include coating 300 to reduced friction or trauma when in use. In some cases, elongated member (e.g., guidewire 10) can include a hydrophilic coating layer on an exterior of a polyurethane extrusion layer on an exterior of metallic core 302 of the guidewire.

In some embodiments, elongated member (e.g., guidewire 10) may include one or more region(s) (as seen in the portion of guidewire 10 being gripped in FIG. 15) in which the jacket and/or outer polymeric layer is stripped, or removed, from metallic core 302 of the guidewire, so as to expose a region of bare metal (e.g., an exposed region 304 of metallic core 302). In some cases, exposed region 304 can be configured so as to interface with a locking and/or gripping mechanism (e.g., such as the twist-lock hub) to be used via a practitioner. In some cases, exposed region 304 may be located adjacent to clip 200. When gripped, or used by a practitioner, such a region can provide a more secure method of engagement between the locking hub and the guidewire. Metal on metal contact can lead to a more secure attachment between locking hub 110 and the elongate member. For example, clip 200 may be metallic and engage with the exposed region 304 that is metallic. This engagement may be more secure than clip 200 engaging with coating 300 of the elongate member or the polymeric cantilever portions 134 and 136 engaging with elongate member.

A length and location of exposed region 304 or region along the length of the guidewire can vary. For instance, in some embodiments, exposed region 304 is positioned along a mid-portion of the elongate body and can span a longitudinal distance of approximately two to 20 centimeters. However, in alternate embodiments, such exposed region 304 may be longer.

As was previously described with respect to FIG. 14E, first cantilever portion 134 and second cantilever portion 136 are separated via gap 142. When forced towards longitudinal axis 140A, first cantilever portion 134 and second cantilever portion 136 deflect such that cantilever heads 135 and 137 displace to axis 140A. This displacement also deflects second portion 214 of first member 210 and second portion 224 of second member 220 toward longitudinal axis 140A. Gripping portions 139A-139B can approach and the interior of second portion 214 of first member 210 and the interior of second portion 224 of second member 220, can contact each other. In such a way, gripping portions 139A-139B can be made to grip guidewire 10 disposed within bore 141 of locking hub 110 and extends through space 143 and gap 142.

In other words, clip 200 clamps down on guidewire 10 as cantilever portions 134 and 136 deflect toward longitudinal axis 140A. The interior of second portion 214 of first member 210 and second portion 224 of second member 220 clamp down on guidewire 10 to secure it in place. In some embodiment, a portion of second portion 214 and third portion 216 of first member 210 and second portion 224 and third portion 226 of second member 220 engage with guidewire 10. In some embodiments, a length of second portion 214 and second portion 224 engage with guidewire 10.

FIG. 16A-16F illustrate the process of coupling first detents 130 of proximal component 120 to second detents 161 of distal component 150. While this description includes clip 200 and first detent 130′. This description is applicable to the locking hub 110 described in FIG. 1-13B, even though some of the components have different geometries.

FIG. 16A-16C illustrate locking hub 110 in an open configuration or first position and FIG. 16D-16F illustrate locking hub 110 in a closed or second position. As illustrated in FIGS. 16A and 16D, proximal component 120 is inserted into distal component 150 and rim 129 has engaged with windows 194 securing proximal component 120 within distal component 150.

FIG. 16A illustrates catheter system 100 in the first position, FIG. 16B illustrates a cross-sectional view along line 16B-16B of the interaction between first detent 130 and second detents 161A in the first position, and FIG. 10C illustrates a cross-sectional view along line 16C-16C of the interaction between cantilever heads 135 and 137 and cavity 166 in the first position. FIG. 16D illustrates catheter system 100 in the second position, FIG. 16E illustrates a cross-sectional view along line 16E-16E of the interaction between first detent 130 and second detents 161B in the second position, and FIG. 16C illustrates a cross-sectional view along line 16C-16C of the interaction between cantilever heads 135 and 137 and cavity 166 in the second position.

FIG. 16B illustrates locking hub 110 in the first position. In the first position, first detents 130 are engaged with second detents 161A. FIG. 16C illustrates cantilever heads 135 and 137 in an unconstrained position. In other words, major diameter 144B of cantilever heads 135 and 137 is similar to major diameter 184 of cavity 166 and minor diameter 144A of cantilever heads 135 and 137 is similar to minor diameter 180 of cavity 166. Major diameter 144B of cantilever head 135 and 137 includes gap 142.

To clamp cantilever heads 135 and 137 onto the elongate member, the user may twist proximal component 120 relative to distal component 150 using flanges 122. As the user twists proximal component 120 to the second position illustrated in FIG. 16D-16F (e.g., a quarter turn), first detents 130 slide along transition portions 164 to second detents 161B. An audible click may indicate that first detents 130 are secured in second detents 161B. A stop 161C ensures that first detents 130 do no slide past second detents 161B.

FIG. 16E illustrates locking hub 110 in the second position. In the first position, first detents 130 are engaged with second detents 161B. FIG. 16F illustrates cantilever heads 135 and 137 in a constrained position. In other words, major diameter 144B of cantilever heads 135 and 137 is diminished and is similar to minor diameter 180 of cavity 166. In the constrained position, gap 142 is diminished or closed so that gripping portion 139A-139B engage the elongate member.

To unclamp cantilever heads 135 and 137 from the elongate member, the user may twist proximal component 120 relative to distal component 150 using flanges 122. As the user twists proximal component 120 to the first position illustrated in FIG. 16A-16C (e.g., a quarter turn), first detents 130 slide along transition portions 164 to second detents 161A. An audible click may indicate that first detents 130 are secured in second detents 161A. A stop 161D ensures that first detents 130 do no slide past second detents 161A.

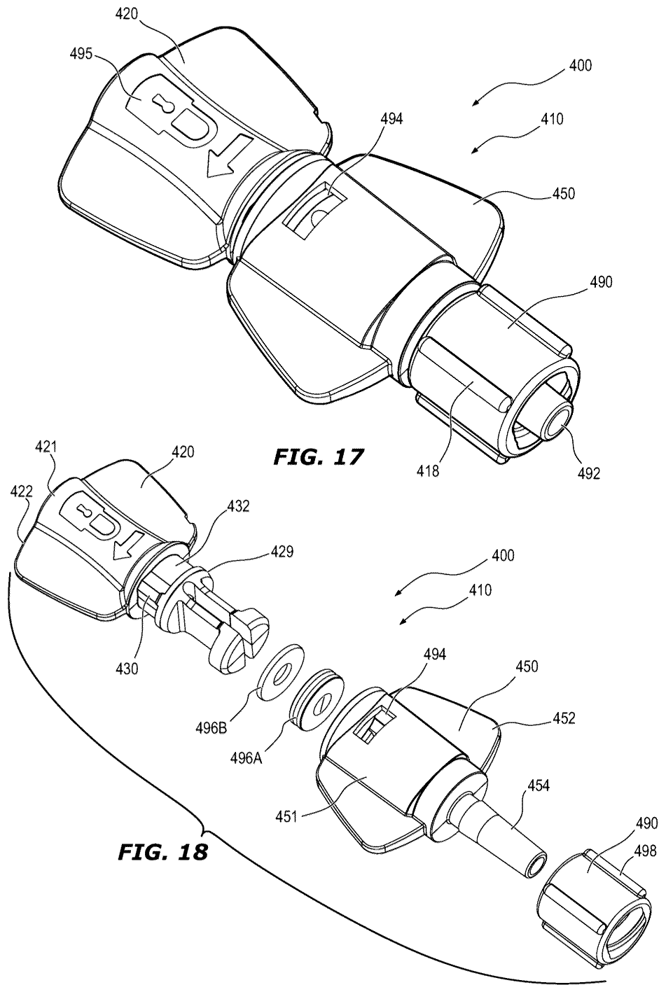

FIG. 17 illustrates a perspective view of a locking hub system 400 according to some embodiments described herein. Locking hub system 400 may include a locking hub 410 with a proximal component 420, a distal component 450, and a Luer lock 490. A bore 492 can pass from a proximal end to a distal end of locking hub system 400. Bore 492 can pass through a longitudinal axis of locking hub system 400, e.g., from a proximal end to a distal end of the locking hub system 400. In some embodiments, an elongate member (e.g., a guidewire) can pass longitudinally through locking hub system 400 through bore 492.

Proximal component 420 of locking hub 410 is similar to proximal component 120 illustrated in FIG. 1-7D. Proximal component 420 can include visible indicia 495 to signal to a user of locking hub system 400 a direction for rotation used to lock locking hub 410. Proximal component 420 can include a main body 421 and flanges 422.

Distal component 450 may be similar to distal component 150 illustrated in FIG. 8-9B. Distal component 450 may further include can include one or more windows 494 which create aspects of a detent means. Proximal component 420 of locking hub 410 may include a plurality of first detents 430. First detents 430 extend radially outward from cylindrical portion 432. For example, proximal component 420 may include cylindrical portion 432 with includes one or more first detents 430. In the illustrated embodiment of FIG. 17, a single first detent 430 is illustrated, but cylindrical portion 432 may include two or more first detents 430. Cylindrical portion 432 may further include a rim 429 that is configured to engage with windows 494 of distal component 450. In other words, as proximal component 420 is inserted into distal component 450, rim 429 engages with windows 494 and secures proximal component 420 to distal component 450. This attachment may be irreversible.

Distal component 450 may further include flanges 452 that can extend radially from a main body 451. Flanges 452 can be used by a user to hold, or secure distal component 450 of locking hub 410 as proximal component 420 is rotated, or torqued (e.g., with respect to locking hub 410 or other distal component 450 of locking hub 410).

Luer lock 490 can be used to fluidly connect locking hub 410 to a medical device. Luer lock 490 can include ridges 498 for enhancing grip and torquability with respect to a user of locking hub 410.

Flanges 452 can extend radially from main body 451. Flanges 452 can be used by a user to hold, or secure distal component 450 of locking hub 410 as proximal component 420 is rotated, or torqued (e.g., with respect to proximal component 420).

FIG. 18 illustrates an exploded view of locking hub system 400, according to some embodiments described herein. Proximal component 420, distal component 450, and Luer lock 490 are visible. As illustrated in FIG. 18, distal component 450 can include a distal portion 454. An end portion 458 can transition from main body 451 and distal portion 454.

In some embodiments, a valve member 496A and a washer 496B can be used to reduce fluid leaking between proximal component 420 and distal component 450.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

### Examples

Example 1. A locking mechanism, comprising: a proximal component comprising: a distal end; and a gap disposed between cantilever heads of the distal end, wherein a first spanning distance comprises the cantilever heads and the gap; and a distal component rotationally coupled with the proximal component, the distal component comprising: a cavity comprising a distal end, the cavity rotationally coupled with the distal end of the proximal component; and a second spanning distance of the distal end of the cavity; wherein a rotation of the proximal component with respect to the distal component, or the distal component with respect to the proximal component, is configured to: align a direction of the first spanning distance with a direction of the second spanning distance; and diminish the gap of the proximal component.

Example 2. The locking mechanism of example 1, wherein the rotation of the proximal component with respect to the distal component is configured to engage or disengage a closed configuration in which the gap is diminished.

Example 3. The locking mechanism of example 1, wherein diminishment of the gap is configured to secure a guidewire to the locking mechanism.

Example 4. The locking mechanism of example 1, wherein the rotation of the proximal component with respect to the distal component, or the distal component with respect to the proximal component, comprises a quarter turn.

Example 5. The locking mechanism of example 1, wherein the distal end of the proximal component comprises an elliptical profile, and wherein the distal end of the cavity of the distal component comprises an elliptical profile.

Example 6. The locking mechanism of example 1, wherein the first spanning distance comprises a major diameter of the distal end of the proximal component, and wherein the second spanning distance comprises a minor diameter of the distal end of the cavity of the distal component.

Example 7. The locking mechanism of example 1, wherein the proximal component comprises a first cantilever portion and a second cantilever portion, wherein alignment of the direction of the first spanning distance with the direction of the second spanning distance deflects the first cantilever portion and the second cantilever portion towards a central longitudinal axis of the proximal component.

Example 8. The locking mechanism of example 7, wherein the first cantilever portion and the second cantilever portion comprise gripping portions configured to secure a guidewire, the gripping portions comprising at least one of: a channel portion; an elastomeric coating; a serrated surface; a beveled surface; or a smooth surface.

Example 9. The locking mechanism of example 7, further comprises a clip comprising a first member, a second member, and a curved member, wherein the clip comprises a second gap between the first member and the second member; wherein the clip is disposed in a space between the first cantilever portion and the second cantilever portion; and wherein the clip is configured to secure a guidewire when the gap is diminished which simultaneously diminishes the second gap.

Example 10. The locking mechanism of example 9, wherein the clip is constrained with the space between the first cantilever portion and the second cantilever portion and prevents longitudinal movement and lateral movement of the clip relative to the proximal component.

Example 11. The locking mechanism of example 9, wherein the second member of the clip comprises a pair of barbs that are configured to secure the clip within the space between the first member and the second member by engaging with a cantilever head of the second cantilever portion; and wherein the barbs prevent longitudinal movement of the clip relative to the proximal component.

Example 12. The locking mechanism of example 9, wherein the curved member of the clip comprises an aperture that surround a ridge of a bore that extends through the proximal component; and wherein an edge of the aperture engages the ridge of the bore and prevents lateral movement of the clip relative to the proximal component.

Example 13. The locking mechanism of example 9, wherein a material of the clip is different from a material of the proximal component.

Example 14. The locking mechanism of example 13, wherein the material of the clip is metal.

Example 15. The locking mechanism of example 9, wherein the clip is slidable into the space between the first cantilever portion and the second cantilever portion and wherein the clip is secured within the space after the clip is slid into the space between the first cantilever portion and the second cantilever portion.

Example 16. The locking mechanism of example 1, wherein the proximal component comprises a radial protrusion configured to engage with a detent of the distal component to maintain a rotational position of the proximal component with respect to the distal component.

Example 17. The locking mechanism of example 16, wherein rotation of the proximal component with respect to the distal component, rotation of the distal component with respect to the proximal component, engagement of the radial protrusion with the detent, or disengagement of the radial protrusion with the detent, provides a visual, tactile, or an auditory feedback to a user of the locking mechanism.

Example 18. A hub, comprising: a proximal component comprising a distal end, wherein the distal end of the proximal component comprises a gap and a first spanning distance; and a distal component rotationally coupled with the proximal component, the distal component comprising a cavity configured to receive the proximal component, the cavity comprising a distal end, wherein the distal end of the cavity comprises a second spanning distance; wherein the first spanning distance is greater than the second spanning distance; and wherein a rotation of the proximal component with respect to the distal component, or the distal component with respect to the proximal component, is configured to align a direction of the first spanning distance with a direction of the second spanning distance such that the gap is diminished.

Example 19. The hub of example 18, wherein the rotation of the proximal component with respect to the distal component is configured to engage or disengage a closed configuration in which the gap is diminished.

Example 20. The hub of example 18, wherein diminishment of the gap is configured to secure a guidewire to the hub.

Example 21. The hub of example 18, wherein the rotation of the proximal component with respect to the distal component, or the distal component with respect to the proximal component, comprises a quarter turn.

Example 22. The hub of example 18, wherein the distal end of the proximal component comprises an elliptical profile, and wherein the distal end of the cavity of the distal component comprises an elliptical profile.

Example 23. The hub of example 18, wherein the first spanning distance comprises a major diameter of the distal end of the proximal component, and wherein the second spanning distance comprises a minor diameter of the distal end of the cavity of the distal component.

Example 24. The hub of example 18, wherein the proximal component comprises a first cantilever portion and a second cantilever portion, wherein alignment of the direction of the first spanning distance with the direction of the second spanning distance deflects the first cantilever portion and the second cantilever portion towards a central longitudinal axis of the hub.

Example 25. The hub of example 24, wherein the first cantilever portion and the second cantilever portion comprise gripping portions configured to secure a guidewire, the gripping portions comprising at least one of: a channel portion; an elastomeric coating; a serrated surface; a beveled surface; or a smooth surface.

Example 26. The hub of example 24, further comprising a metal clip that is disposed within a space between the first cantilever portion and the second cantilever portion and is secured within the space but not fixed to the first cantilever portion and the second cantilever portion.

Example 27. The hub of example 26, wherein the metal clip further comprises an aperture that is configured to allow a guidewire to extend through the clip and the clip is configured to clamp onto the guidewire to secure the guidewire.

Example 28. The hub of example 18, wherein the proximal component comprises a radial protrusion configured to engage with a detent of the distal component to maintain a rotational position of the proximal component with respect to the distal component.

Example 29. The hub of example 28, wherein rotation of the proximal component with respect to the distal component, rotation of the distal component with respect to the proximal component, engagement of the radial protrusion with the detent, or disengagement of the radial protrusion with the detent, provides a visual, tactile, or an auditory feedback to a user of the hub.

Example 30. A system, comprising: a hub, comprising: a proximal component comprising a distal end, wherein the distal end of the proximal component comprises a gap and a first spanning distance; and a distal component rotationally coupled with the proximal component, the distal component comprising a cavity configured to receive the proximal component, the cavity comprising a distal end, wherein the distal end of the cavity comprises a second spanning distance; wherein the first spanning distance is greater than the second spanning distance; and an elongate member passing through the gap of the proximal component; wherein a rotation of the proximal component with respect to the distal component, or the distal component with respect to the proximal component, is configured to align a direction of the first spanning distance and a direction of the second spanning distance such that the gap is diminished and the elongate member is secured to the hub.

Example 31. The system of example 30, wherein the rotation of the proximal component with respect to the distal component is configured to engage or disengage a closed configuration in which the gap is diminished.

Example 32. The system of example 30, wherein the rotation of the proximal component with respect to the distal component, or the distal component with respect to the proximal component, comprises a quarter turn.

Example 33. The system of example 30, wherein the proximal component comprises a first cantilever portion and a second cantilever portion, wherein alignment of the direction of the first spanning distance with the direction of the second spanning distance deflects the first cantilever portion and the second cantilever portion towards a central longitudinal axis of the hub.

Example 34. The system of example 33, wherein the first cantilever portion and the second cantilever portion comprise gripping portions configured to secure a guidewire, the gripping portions comprising at least one of: a channel portion; an elastomeric coating; a serrated surface; a beveled surface; or a smooth surface.

Example 35. The hub of example 33, further comprising a metal clip that is disposed within a space between the first cantilever portion and the second cantilever portion and is secured within the space; and wherein the metal clip further comprises an aperture that is configured to allow the elongate member extend through the clip and the clip is clampable onto the elongate member to secure the elongate member.

Example 36. The hub of example 35, wherein the elongate member comprises a core and a coating, wherein the elongate member comprises an exposed region in which core does not have a coating, and wherein the clip is configured to engage the elongate member at the exposed region.

Example 37. The system of example 30, wherein the proximal component comprises a radial protrusion configured to engage with a detent of the distal component to maintain a rotational position of the proximal component with respect to the distal component.

Example 38. The system of example 37, wherein rotation of the proximal component with respect to the distal component, rotation of the distal component with respect to the proximal component, engagement of the radial protrusion with the detent, or disengagement of the radial protrusion with the detent, provides a visual, tactile, or an auditory feedback to a user of the hub.

Example 39. A locking mechanism, comprising: a proximal component comprising: a distal end; and a gap disposed between cantilever heads of the distal end, wherein a first spanning distance comprises the cantilever heads and the gap; and a distal component rotationally coupled with the proximal component, the distal component comprising a cavity configured to receive the distal end of the proximal component; wherein a rotation of the distal end of the proximal component within the cavity of the distal component, or the cavity of the distal component with respect to the proximal component, is configured to diminish the gap between the cantilever heads of the proximal component.

Example 40. The locking mechanism of example 39, wherein the rotation of the distal end of the proximal component within the cavity of the distal component, or the cavity of the distal component with respect to the proximal component, is configured to align a direction of a first spanning distance of the distal end of the proximal component with a direction of a second spanning distance of a distal end of the cavity of the distal component.

1. A locking mechanism, comprising:
a proximal component comprising:
a distal end; and
a gap disposed between cantilever heads of the distal end, wherein a first spanning distance comprises the cantilever heads and the gap; and
a distal component rotationally coupled with the proximal component, the distal component comprising:
a cavity comprising a distal end, the cavity rotationally coupled with the distal end of the proximal component; and
a second spanning distance of the distal end of the cavity;
wherein a rotation of the proximal component with respect to the distal component, or the distal component with respect to the proximal component, is configured to:
align a direction of the first spanning distance with a direction of the second spanning distance; and
diminish the gap of the proximal component.

2. The locking mechanism of claim 1, wherein the rotation of the proximal component with respect to the distal component is configured to engage or disengage a closed configuration in which the gap is diminished.

3. The locking mechanism of claim 1, wherein the rotation of the proximal component with respect to the distal component, or the distal component with respect to the proximal component, comprises a quarter turn.

4. The locking mechanism of claim 1, wherein the proximal component comprises a first cantilever portion and a second cantilever portion, wherein alignment of the direction of the first spanning distance with the direction of the second spanning distance deflects the first cantilever portion and the second cantilever portion towards a central longitudinal axis of the proximal component.

5. The locking mechanism of claim 4, further comprises a clip comprising a first member, a second member, and a curved member, wherein the clip comprises a second gap between the first member and the second member;
wherein the clip is disposed in a space between the first cantilever portion and the second cantilever portion; and
wherein the clip is configured to secure a guidewire when the gap is diminished which simultaneously diminishes the second gap.

6. The locking mechanism of claim 5, wherein the clip is constrained with the space between the first cantilever portion and the second cantilever portion and prevents longitudinal movement and lateral movement of the clip relative to the proximal component.

7. The locking mechanism of claim 5, wherein the second member of the clip comprises a pair of barbs that are configured to secure the clip within the space between the first member and the second member by engaging with a cantilever head of the second cantilever portion; and
wherein the barbs prevent longitudinal movement of the clip relative to the proximal component.

8. The locking mechanism of claim 5, wherein the curved member of the clip comprises an aperture that surround a ridge of a bore that extends through the proximal component; and
wherein an edge of the aperture engages the ridge of the bore and prevents lateral movement of the clip relative to the proximal component.

9. The locking mechanism of claim 1, wherein the proximal component comprises a radial protrusion configured to engage with a detent of the distal component to maintain a rotational position of the proximal component with respect to the distal component.

10. A hub, comprising:
a proximal component comprising a distal end, wherein the distal end of the proximal component comprises a gap and a first spanning distance; and
a distal component rotationally coupled with the proximal component, the distal component comprising a cavity configured to receive the proximal component, the cavity comprising a distal end, wherein the distal end of the cavity comprises a second spanning distance;
wherein the first spanning distance is greater than the second spanning distance; and
wherein a rotation of the proximal component with respect to the distal component, or the distal component with respect to the proximal component, is configured to align a direction of the first spanning distance with a direction of the second spanning distance such that the gap is diminished.

11. The hub of claim 10, wherein the rotation of the proximal component with respect to the distal component is configured to engage or disengage a closed configuration in which the gap is diminished.

12. The hub of claim 10, wherein the distal end of the proximal component comprises an elliptical profile, and wherein the distal end of the cavity of the distal component comprises an elliptical profile.

13. The hub of claim 10, wherein the first spanning distance comprises a major diameter of the distal end of the proximal component, and wherein the second spanning distance comprises a minor diameter of the distal end of the cavity of the distal component.

14. The hub of claim 10, wherein the proximal component comprises a first cantilever portion and a second cantilever portion, wherein alignment of the direction of the first spanning distance with the direction of the second spanning distance deflects the first cantilever portion and the second cantilever portion towards a central longitudinal axis of the hub.

15. The hub of claim 14, further comprising a metal clip that is disposed within a space between the first cantilever portion and the second cantilever portion and is secured within the space but not fixed to the first cantilever portion and the second cantilever portion.

16. A system, comprising:
a hub, comprising:
a proximal component comprising a distal end, wherein the distal end of the proximal component comprises a gap and a first spanning distance; and
a distal component rotationally coupled with the proximal component, the distal component comprising a cavity configured to receive the proximal component, the cavity comprising a distal end, wherein the distal end of the cavity comprises a second spanning distance;
wherein the first spanning distance is greater than the second spanning distance; and
an elongate member passing through the gap of the proximal component;
wherein a rotation of the proximal component with respect to the distal component, or the distal component with respect to the proximal component, is configured to align a direction of the first spanning distance and a direction of the second spanning distance such that the gap is diminished and the elongate member is secured to the hub.

17. The system of claim 16, wherein the rotation of the proximal component with respect to the distal component is configured to engage or disengage a closed configuration in which the gap is diminished.

18. The system of claim 16, wherein the proximal component comprises a first cantilever portion and a second cantilever portion, wherein alignment of the direction of the first spanning distance with the direction of the second spanning distance deflects the first cantilever portion and the second cantilever portion towards a central longitudinal axis of the hub.

19. The hub of claim 18, further comprising a metal clip that is disposed within a space between the first cantilever portion and the second cantilever portion and is secured within the space; and
wherein the metal clip further comprises an aperture that is configured to allow the elongate member to extend through the clip and the clip is clampable onto the elongate member to secure the elongate member.

20. The hub of claim 19, wherein the elongate member comprises a core and a coating, wherein the elongate member comprises an exposed region in which core does not have a coating, and wherein the clip is configured to engage the elongate member at the exposed region.
